(19) United States
(12) Patent Application Publication
(10) Pub. No.: US 2002/0157143 A1
(43) Pub. Date: Oct. 24, 2002

(54) Soybean plants with enhanced yields and methods for breeding for and screening of soybean plants with enhanced yields

(76) Inventors: Vergel C. Concibido, Maryland Heights, MO (US); Xavier Delannay, Defiance, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/037,598

(22) Filed: Jan. 4, 2002

Related U.S. Application Data
(60) Provisional application No. 60/260,040, filed on Jan. 5, 2001.

(51) Int. Cl. A01H 5/00

(52) U.S. Cl. 800/312

(57) ABSTRACT

The present invention is in the field of plant breeding and genetics, particularly as it pertains to Glycine max (soybean). More specifically, the invention relates to a quantitative trait loci that is associated with enhanced yield in Glycine max, Glycine max having such loci and methods for breeding for and screening of Glycine max with such loci. The invention further relates to the use of exotic germplasm in a breeding program.

This application claims the benefit of U.S. Provisional Application No. 60/260,040, filed Jan. 5, 2001.

### FIELD OF THE INVENTION

The present invention is in the field of plant breeding and genetics, particularly as it pertains to Glycine max (soybean). More specifically, the invention relates to alleles of a quantitative trait locus that are associated with enhanced yield in Glycine max, Glycine max plants having such alleles and methods for breeding for and screening of Glycine max plants with such alleles. The invention further relates to the use of exotic Glycine max germplasm in a breeding program.

### BACKGROUND OF THE INVENTION

The soybean, Glycine max (L.) Merril (Glycine max or soybean), is one of the major economic crops grown worldwide as a primary source of vegetable oil and protein (Sinclair and Backman, Compendium of Soybean Diseases, 3^{rd} Ed. APS Press, St. Paul, Minn., p. 106. (1989), the entirety of which is herein incorporated by reference). The growing demand for low cholesterol and high fiber diets has also increased soybean's importance as a health food.

Prior to 1940, soybean cultivars were either direct releases of introductions brought from Asia or pure line selections from genetically diverse plant introductions. The soybean plant was primarily used as a hay crop in the early part of the 19th century. Only a few introductions were large-seeded types useful for feed grain and oil production. From the mid 1930's through the 1960's, gains in soybean seed yields were achieved by changing the breeding method from evaluation and selection of introduced germplasm to crossing elite by elite lines. The continuous cycle of cross hybridizing the elite strains selected from the progenies of previous crosses resulted in the modern day cultivars.

Over 10,000 soybean strains have now been introduced into the United States since the early 1900's (Bernard et al., United States National Germplasm Collections. In: L. D. Hil (ed.), World Soybean Research, pp. 286-289. Interstate Printers and Publ., Danville, Ill. (1976), the entirety of which is herein incorporated by reference). A limited number of those introductions form the genetic base of cultivars developed from the hybridization and selection programs (Johnson and Bernard, The Soybean, Norman Ed., Academic Press, N.Y. pp. 1-73 (1963)). For example, in a survey conducted by Specht and Williams, Genetic Contributions, Fehr eds. American Soil Association, Wisconsin, pp. 49-73 (1984), for the 136 cultivars released from 1939 to 1989, only 16 different introductions were the source of cytoplasm for 121 of that 136.

Six introductions, ‘Mandarin,’ ‘Manchu,’ ‘Mandarin’ (Ottawa), “Richland,’ ‘AK’ (Harrow), and ‘Mukden,’ contributed nearly 70% of the germplasm represented in 136 cultivar releases. To date, modern day cultivars can be traced back from these six soybean strains from southern China. In a study conducted by Cox et al., Crop Sci. 25:529-532 (1988), the soybean germplasm is comprised of 90% adapted materials, 9% unadapted, and only 1% from exotic species.

Marker assisted introgression of traits into plants has been reported. Marker assisted introgression involves the transfer of a chromosome region defined by one or more markers from one germplasm to a second germplasm. An initial step in that process is the localization of the trait by gene mapping. Gene mapping studies to analyze agronomic traits have been reported in many plants including Glycine max and Glycine max x Glycine soja. Gene mapping is the process of determining a gene's position relative to other genes and genetic markers through linkage analysis. The basic principle for linkage mapping is that the closer together two genes are on the chromosome, the more likely they are to be inherited together (Rothwell, Understanding Genetics. 4^{th} Ed. Oxford University Press, New York, p. 703 (1988), the entirety of which is herein incorporated by reference). Briefly, a cross is made between two genetically compatible but divergent parents relative to traits under study. Genetic markers are then used to follow the segregation of traits under study in the progeny from the cross (often a backcross, F₂, or recombinant inbred population).

Linkage analysis is based on the level at which markers and genes are co-inherited (Rothwell, Understanding Genetics. 4^{th} Ed. Oxford University Press, New York, p. 703 (1988)). Statistical tests like chi-square analysis can be used to test the randomness of segregation or linkage (Kochert, The Rockefeller Foundation International Program on Rice Biotechnology, University of Georgia Athens, Ga., pp. 1-14 (1989), the entirety of which is herein incorporated by reference). In linkage mapping, the proportion of recombinant individuals out of the total mapping population provides the information for determining the genetic distance between the loci (Young, Encyclopedia of Agricultural Science, Vol. 3, pp. 275-282 (1994), the entirety of which is herein incorporated by reference).

Classical mapping studies utilize easily observable, visible traits instead of molecular markers. These visible traits are also known as naked eye polymorphisms. These traits can be morphological like plant height, fruit size, shape and color or physiological like disease response, photoperiod sensitivity or crop maturity. Visible traits are useful and are still in use because they represent actual phenotypes and are easy to score without any specialized lab equipment. By contrast, the other types of genetic markers are arbitrary loci for use in linkage mapping and often not associated to specific plant phenotypes (Young, Encyclopedia of Agricultural Science, Vol. 3, pp. 275-282 (1994)). Many morphological markers cause such large effects on phenotype that they are undesirable in breeding programs. Many other visible traits have the disadvantage of being developmentally regulated (i.e. expressed only certain stages; or at specific tissue and organs). Oftentimes, visible traits mask the effects of linked minor genes making it nearly impossible to identify desirable linkages for selection (Tanksely, et al., Biotech. 7:257-264 (1989), the entirety of which is herein incorporated by reference).

Although a number of important agronomic characters are controlled by loci having major effects on phenotype, many economically important traits, such as yield and some forms of disease resistance, are quantitative in nature. This type of phenotypic variation in a trait is typically characterized by continuous, normal distribution of phenotypic values in a particular population (Beckmann and Soller, Oxford Surveys of Plant Molecular Biology, Miffen. (ed.), Vol. 3, Oxford University Press, UK., pp. 196-250 (1986), the entirety of which is herein incorporated by reference). Loci contributing to such genetic variation are thought to be minor genes, as opposed to major genes with large effects that follow a Mendelian pattern of inheritance. Individual loci controlling polygenic traits are also predicted to follow a Mendelian type of inheritance, however the contribution of each locus is expressed as an increase or decrease in the final trait value.

The advent of DNA markers, such as restriction fragment length polymorphism markers (RFLPs), microsatellite markers (SSR), single nucleotide polymorphic markers (SNPs), and random amplified polymorphic DNA markers (RAPDs), allow the resolution of complex, multigenic traits into their individual Mendelian components (Paterson et al, Nature 335:721-726 (1988), the entirety of which is herein incorporated by reference). A number of applications of RFLPs and other markers have been suggested for plant breeding. Among the potential applications for RFLPs and other markers in plant breeding include: varietal identification (Soller and Beckmann, Theor. Appl. Genet. 67:25-33 (1983), the entirety of which is herein incorporated by reference; Tanksley et al., Biotech. 7:257-264 (1989), QTL mapping (Edwards et al., Genetics 116:113-115 (1987), the entirety of which is herein incorporated by reference); Nienhuis et al., Crop Sci. 27:797-803 (1987); Osborn et al., Theor. Appl. Genet. 73:350-356 (1987); Romero-Severson et al, Use of RFLPs In Analysis Of Quantitative Trait Loci In Maize, InHelentjaris and Burr (eds.), pp. 97-102 (1989), the entirety of which is herein incorporated by reference; Young et al., Genetics 120:579-585 (1988), the entirety of which is herein incorporated by reference; Martin et al., Science 243:1725-1728 (1989), the entirety of which is herein incorporated by reference); Sarfatti et al., Theor. Appl. Genet. 78:22-26 (1989), the entirety of which is herein incorporated by reference; Tanksley et al., Biotech. 7:257-264 (1989); Barone et al., Mol. Gen. Genet. 224:177-182 (1990), the entirety of which is herein incorporated by reference); Jung et al., Theor. Appl. Genet. 79:663-672 (1990), the entirety of which is herein incorporated by reference; Keim et al., Genetics 126:735-742 (1990), the entirety of which is herein incorporated by reference, Keim et al., Theor. Appl. Genet. 79:465-369 (1990), the entirety of which is herein incorporated by reference; Paterson et al., Genetics 124:735-742 (1990), the entirety of which is herein incorporated by reference; Martin et al., Proc. Natl. Acad. Sci. U.S.A. 88:2336-2340 (1991), the entirety of which is herein incorporated by reference; Messeguer et al., Theor. Appl. Genet. 82:529-536 (1991), the entirety of which is herein incorporated by reference; Michelmore et al., Proc. Natl. Acad. Sci. U.S.A. 88:9828-9832 (1991), the entirety of which is herein incorporated by reference; Ottaviano et al., Theor. Appl. Genet. 81:713-719 (1991), the entirety of which is herein incorporated by reference; Yu et al., Theor. Appl. Genet. 81:471-476 (1991), the entirety of which is herein incorporated by reference; Diers et al., Crop Sci. 32:377-383 (1992), the entirety of which is herein incorporated by reference; Doebley et al., Proc. Natl. Acad. Sci. U.S.A. 87:9888-9892 (1990), the entirety of which is herein incorporated by reference, screening genetic resource strains for useful quantitative trait alleles and introgression of these alleles into commercial varieties (Beckmann and Soller, Theor. Appl. Genet. 67:35-43 (1983), the entirety of which is herein incorporated by reference; Tanksley et al., Biotech. 7:257-264 (1989), marker-assisted selection (Tanksley et al., Biotech. 7:257-264 (1989) and map-based cloning (Tanksley et al., Biotech. 7:257-264 (1989)). In addition, DNA markers can be used to obtain information about: (1) the number, effect, and chromosomal location of each gene affecting a trait; (2) effects of multiple copies of individual genes (gene dosage); (3) interaction between/among genes controlling a trait (epistasis); (4) whether individual genes affect more than one trait (pleiotropy); and (5) stability of gene function across environments (G×E interactions).

Gene mapping studies associated with QTLs, have focused on agronomic and morphological characters in plants. In maize (Zea mays L.), QTLs contributing to heterosis in several quantitative traits have been mapped (Stuber et al., Genetics 132:823-839 (1992), the entirety of which is herein incorporated by reference, as well as QTLs for heat tolerance (Ottaviano et al., Theor. Appl. Genet. 81:713-719 (1991) and morphological characters distinguishing maize from teosinte (Zea mays ssp. mexicana) (Doebley et al., Proc. Natl. Acad. Sci. (U.S.A.) 87:9888-9892 (1990). In tomato, RFLPs have been used in locating and determining effects of QTLs associated with fruit size, pH, soluble solids (Paterson et al., Genetics 124:735-742 (1990) and water use efficiency (Martin et al, Genetics 120:579-585 (1989).

Tanksley et al. suggested the use of molecular markers to introduce QTLs from exotic germplasm (Tanksley et al., Theor. Appl. Genet. 92: 191-203 (1996). Paterson et al., report the location of putative QTLs in an F₂ population that results from a cross between a domestic tomato strain and an exotic relative (Paterson et al., Genetics 127: 181-197 (1991). The present effort evolved from efforts to locate and introduce traits that enhance agronomical traits into Glycine max from Glycine max introductions. Activities not described by Tanksley et al., Theor. Appl. Genet. 92: 191-203 (1996) or Paterson et al., Genetics 127: 181-197 (1991). Lark et al. Proc. Natl. Acad. Sci. USA 92:4656-4660 (1995) described the interaction of two genetic loci in soybean PI290136 that contribute to height and yield. One of these loci was tightly linked to a black seeded (black seed coat) trait. The black seeded trait is undesirable in soybean for most agricultural markets. In order for any of these loci to be agronomically useful, this linkage would have to be genetically broken and yellow seed coat soybean plants produced.

The present invention provides high yielding Glycine max plants having yellow coat seeds and methods for producing such plants that address the following difficulties: (A) the introgression of a single loci high yield trait into agronomically useful Glycine max varieties; and (B) breaking the genetic linkage of the high yield loci with the black seed color present in Glycine max PI290136.

### SUMMARY OF THE INVENTION

The present invention provides a method of soybean breeding for a yellow seed coat Glycine max plant having enhanced yield comprising: (A) crossing a black seed coat Glycine max PI290136 parent plant or progeny thereof with a yellow seed coat Glycine max parent plant to produce a segregating population of progeny plants; and (B) screening the segregating population of progeny plants for the presence of a DNA molecular marker of a sufficient length that is homologous or complementary to a DNA molecule selected from the group consisting of SEQ ID NO: 19-37, wherein a member of the progeny plants has an enhanced yield allele (SY5) derived from the Glycine max PI290136 plant and that maps to linkage group U03 of the Glycine max PI290136 plant; and (C) selecting the member plant for further crossing and selection, wherein the member plant selected has a yellow seed coat and enhanced yield relative to the yellow seed coat Glycine max parent plant.

The present invention includes and provides a yellow seeded (yellow seed coat) Glycine max plant having an allele of a quantitative trait locus associated with enhanced yield in the Glycine max plant. In one embodiment, the present invention includes and provides a yellow seed coat Glycine max plant having an allele of a quantitative trait locus associated with enhanced yield in the Glycine max plant wherein the yellow seed coat Glycine max plant is provided in a seed deposit to the American Type Culture Collection #PTA-2323.

The present invention further provides for the soybean seed having ATCC Accession No. PTA-2323, and a soybean plant or its parts produced by growing the seed of PTA-2323. The reproductive parts, especially the pollen and ovules of PTA-2323 plants and progeny thereof is an aspect of the invention. The progeny of a cross between a first soybean plant and a second soybean plant, wherein the first soybean plant has at least one ancestor derived from PTA-2323 and has a yellow seed coat and enhanced yield.

The present invention also provides an elite yellow seeded Glycine max plant cultivar comprising an allele of an enhanced yield quantitative trait locus derived from a Glycine max PI290136 plant or progeny thereof, wherein the enhanced yield quantitative trait locus is located on linkage group U03 of a black seed coat Glycine max PI290136 and linked to a DNA molecular marker derived from and complementary to Satt187 (SEQ ID NO:20), Sat_{—}212, Sat_{—}215, Sy50 (SEQ ID NO:22), SCNB190 (SEQ ID NO:25), SCNB188 (SEQ ID NO:21), SAHH (SEQ ID NO:26), SCNB187 (SEQ ID NO:23), XET1 (SEQ ID NO:27), Sy36 (SEQ ID NO:24), Satt315 (SEQ ID NO: 19), and chalcone synthase gene cluster (SEQ ID NO:28-37).

The present invention also provides a yellow seeded Glycine max plant comprising a DNA molecule, wherein the DNA molecule has a substantially homologous sequence as DNA found in an allele of the enhanced yield quantitative trait locus derived from Glycine max PI290136 or progeny thereof and located on linkage group U03, and linked to a DNA molecular marker derived from and complementary to Satt187, Sat_{—}212, Sat_{—}215 Sy50, SCNB190, SCNB188, SAHH, SCNB187, XET1, Sy36, Satt315 and the chalcone synthase gene cluster.

The present invention also provides a yellow seeded Glycine max seed from a Glycine max plant comprising DNA of an allele of a quantitative trait locus for enhanced yield, wherein the DNA is substantially homologous to at least one DNA molecule selected from the group consisting of SEQ ID NO: 1-37.

The present invention also provides a container of over 40,000 yellow seeded Glycine max seeds, wherein over 80% of the seeds have an allele of the quantitative trait locus associated with enhanced yield in the Glycine max plant, wherein the allele of the enhanced yield quantitative trait locus is also located on linkage group U03 of a Glycine max PI290136 plant and associated with the DNA molecular markers derived from and complementary to Satt187, Sat_{—}212, Sat_{—}215 Sy50, SCNB190, SCNB188, SAHH, SCNB187, XET1, Sy36, Satt315 and the chalcone synthase gene cluster.

The present invention also provides a progeny yellow seeded Glycine max plant containing an enhanced yield quantitative trait locus located on linkage group U03 of a Glycine max PI290136 plant and associated with the DNA molecular markers derived from and complementary to Satt187, Sat_{—}212, Sat_{—}215 Sy50, SCNB190, SCNB188, SAHH, SCNB187, XET1, Sy36, Satt315 and the chalcone synthase gene cluster, which exhibits an enhanced yield compared to a yellow seeded Glycine max first parent plant that does not contain the enhanced yield quantitative trait locus. The progeny yellow seeded Glycine max plant comprising a genome homozygous or heterozygous with respect to a genetic allele that is native to a second parent plant selected from the group consisting of Glycine max PI290136 and progeny thereof, and the genetic allele is non-native to the first parent plant.

The present invention also provides a method for determining the likelihood of a quantitative trait allele located on linkage group U03 of a Glycine max PI290136 plant for enhanced yield in a yellow seed coat Glycine max plant comprising the steps of: (A) obtaining mRNA from the yellow seed coat Glycine max plant; (B) detecting a mRNA transcript molecule (C) determining the presence or absence of the mRNA molecule relative to mRNA obtained from a sibling yellow seed coat Glycine max plant not containing the quantitative trait allele derived from a cross with Glycine max PI290136, wherein the presence or absence of the mRNA molecule is indicative of the quantitative trait allele for enhanced yield.

The present invention also provides a method for determining the likelihood of a quantitative trait allele located on linkage group U03 of a Glycine max PI290136 plant for enhanced yield in a cross with a yellow seed coat Glycine max plant comprising the steps of: (A) obtaining mRNA from the progeny of the cross; (B) detecting mRNA transcript molecules; (C) determining the increase or decrease of the level of mRNA molecules, wherein the increase or decrease of the level of mRNA molecules is indicative of the quantitative trait allele for enhanced yield.

The present invention provides and includes a method for the production of a yellow seeded Glycine max elite plant having an enhanced yield quantitative trait allele comprising: (A) crossing a first soybean plant provided in ATCC seed deposit #PTA-2323 or progeny thereof having an enhanced yield quantitative trait allele with a second soybean plant having elite germplasm traits; (B) screening the segregating population for a member having the enhanced yield quantitative trait allele and the elite germplasm traits; (C) selecting the member for further crossing and selection; (D) bulking up seed from said member; and (E) packaging said seed in a container.

The present invention provides for a method of providing an isolated DNA molecule containing an allele of an enhanced yield QTL comprising: (A) constructing a library of soybean genomic DNA selected from the group consisting of Glycine max PI290136 and Glycine max having ATCC Acession No. PTA-2323 containing the enhanced yield QTL; (B) hybridizating the library of soybean genomic DNA with a DNA sequence selected from the group consisting of SEQ ID NO: 19-37; (C) isolating the genomic DNA that hybridizes to the DNA sequence; (D) sequencing the isolated genomic DNA and constructing a contig of sequences; (E) comparing the contig to a soybean genomic DNA sequence not containing the QTL; (F) identifying the polymorphisms in the contig; (G) constructing a plant transformation vector containing the identified polymorphisms; (H) transforming plant cells with the plant transformation vector; (I) regenerating the plant cells into plants; and (J) screening said plants for the enhanced yield phenotype.

The present invention provides for a transformed plant comprising an enhanced yield QTL isolated from Glycine max PI290136, wherein the enhanced yield QTL is located on linkage group U03 of Glycine max PI290136.

### DETAILED DESCRIPTION OF THE INVENTION

The present invention provides a yellow seeded Glycine max plant having an allele of a quantitative trait locus (QTL) associated with enhanced yield in the Glycine max plant, wherein the allele of a quantitative trait locus is also located on linkage group U03 of a Glycine max PI290136 plant associated with molecular markers: Satt187, Sat_{—}212, Sat_{—}215, Sy50, SCNB190, SCNB188, SCNB187, Sy36, Satt315, SAHH, XET1, the visual marker seed coat color, and the chalcone synthase gene cluster, wherein the DNA sequences of these genes are useful as markers for the enhanced yield locus.

The enhanced yield QTL located on linkage group U03 of a black seed coat Glycine max PI290136 plant and on linkage group U03 of a yellow seed coat Glycine max plant ATCC deposit #PTA-2323 is herein referred to as “Sy5”.

A Glycine max plant of the present invention is any yellow seed coat Glycine max plant. In a preferred embodiment, a Glycine max plant of the present invention is an elite plant. An “elite line” is any line that has resulted from breeding and selection for superior agronomic performance. Examples of elite lines are lines that are commercially available to farmers or soybean breeders such as HARTZ™ variety H4994, HARTZ™ variety H5218, HARTZ™ variety H5350, HARTZ™ variety H5545, HARTZ™ variety H5050, HARTZ™ variety H5454, HARTZ™ variety H5233, HARTZ™ variety H5488, HARTZ™ variety HLA572, HARTZ™ variety H6200, HARTZ™ variety H6104, HARTZ™ variety H6255, HARTZ™ variety H6586, HARTZ™ variety H6191, HARTZ™ variety H7440, HARTZ™ variety H4452 Roundup Ready™, HARTZ™ variety H4994 Roundup Ready™, HARTZ™ variety H4988 Roundup Ready™, HARTZ™ variety H5000 Roundup Ready™, HARTZ™ variety H5147 Roundup Ready™, HARTZ™ variety H5247 Roundup Ready™, HARTZ™ variety H5350 Roundup Ready™, HARTZ™ variety H5545 Roundup Ready™, HARTZ™ variety H5855 Roundup Ready™, HARTZ™ variety H5088 Roundup Ready™, HARTZ™ variety H5164 Roundup Ready™, HARTZ™ variety H5361 Roundup Ready™, HARTZ™ variety H5566 Roundup Ready™, HARTZ™ variety H5181 Roundup Ready™, HARTZ™ variety H5889 Roundup Ready™, HARTZ™ variety H5999 Roundup Ready™, HARTZ™ variety H6013 Roundup Ready™, HARTZ™ variety H6255 Roundup Ready™, HARTZ™ variety H6454 Roundup Ready™, HARTZ™ variety H6686 Roundup Ready™, HARTZ™ variety H7152 Roundup Ready™, HARTZ™ variety H7550 Roundup Ready™, HARTZ™ variety H8001 Roundup Ready™ (HARTZ SEED, Stuttgart, Ark., USA); A0868, AG0901, A1553, A1900, AG1901, A1923, A2069, AG2101, AG2201, A2247, AG2301, A2304, A2396, AG2401, AG2501, A2506, A2553, AG2701, AG2702, AG2703, A2704, A2833, A2869, AG2901, AG2902, AG2905, AG3001, AG3002, A3204, A3237, A3244, AG3301, AG3302, A3404, A3469, AG3502, AG3503, A3559, AG3601, AG3701, AG3704, AG3750, A3834, AG3901, A3904, A4045 AG4301, A4341, AG4401, AG4501, AG4601, AG4602, A4604, AG4702, AG4901, A4922, AG5401, A5547, AG5602, A5704, AG5801, AG5901, A5944, A5959, AG6101, AJW2600COR, FPG26932, QR4459 and QP4544 (Asgrow Seeds, Des Moines, Iowa, USA); DKB26-52, DKB28-51, DKB32-52, DKB35-51 and DeKalb variety CX445 (DeKalb, Ill., USA); 91B91, 92B24, 92B37, 92B63, 92B71, 92B74, 92B75, 92B91, 93B01, 93B11, 93B26, 93B34, 93B35, 93B41, 93B45, 93B51, 93B53, 93B66, 93B81, 93B82, 93B84, 94B01, 94B32, 94B53, 95B71, 95B95, 9306, 9294, and 9344 (Pioneer Hi-bred International, Johnstonville, Iowa, USA). An elite plant is any plant from an elite line.

The Sy5 quantitative trait locus of the present invention may be introduced into an elite Glycine max transgenic plant that contains one or more genes for herbicide tolerance, increased yield, insect control, fungal disease resistance, virus resistance, nematode resistance, bacterial disease resistance, mycoplasma disease resistance, modified oils production, high oil production, high protein production, germination and seedling growth control, enhanced animal and human nutrition, low raffinose, environmental stress resistant, increased digestibility, industrial enzymes, pharmaceutical proteins, peptides and small molecules, improved processing traits, improved flavor, nitrogen fixation, hybrid seed production, reduced allergenicity, biopolymers, and biofuels among others. These agronomic traits can be provided by the methods of plant biotechnology as transgenes in Glycine max. It is further understood that a Glycine max plant of the present invention may exhibit the characteristics of any maturity group. The yield enhancing effect of the Sy5 locus in a yellow seed coat phenotype can vary based on the parental genotype (elite line) and on the environmental conditions in which the yield effect is measured. It is within the skill of those in the art of plant breeding and without undue experimentation to use the methods described herein to select from a populaton of plants or from a collection of parental genotypes those that when containing the Sy5 locus result in enhanced yield relative to the parent genotype.

In a preferred embodiment, the nuclear genetic contribution of an exotic black seed coat Glycine max to a yellow seed coat Glycine max of the present invention is less than about 25%. In a more preferred embodiment, the nuclear genetic contribution of an exotic black seed coat Glycine max to a yellow seed coat Glycine max of the present invention is less than about 12.5%. In an even more preferred embodiment, the nuclear genetic contribution of an exotic black seed coat Glycine max to a yellow seed coat Glycine max of the present invention is less than about 6.25%. The an exotic black seed coat Glycine max genetic contribution in a yellow seed coat Glycine max plant of the present invention can be reduced by backcrossing the progeny of a yellow seed coat Glycine max x an exotic black seed Glycine max cross (or progeny thereof) with, for example, a yellow seed coat Glycine max recurrent parent. It is further understood that a yellow seed coat Glycine max plant of the present invention may exhibit the characteristics of any maturity group.

A number of molecular genetic maps of Glycine have been reported (Mansur et al., Crop Sci. 36: 1327-1336 (1996), the entirety of which is herein incorporated by reference; Shoemaker et al., Genetics 144: 329-338 (1996), the entirety of which is herein incorporated by reference; Shoemaker et al., Crop Science 32: 1091-1098 (1992), the entirety of which is herein incorporated by reference; Shoemaker et al., Crop Science 35: 436-446 (1995), the entirety of which is herein incorporated by reference; Tinley and Rafalski, J. Cell Biochem. Suppl. 4E: 291 (1990), the entirety of which is herein incorporated by reference); Cregan et al., Crop Science 39:1464-1490 (1999), the entirety of which is herein incorporated by reference). Glycine max, Glycine soja and Glycine max x. Glycine soja share linkage groups (Shoemaker et al., Genetics 144: 329-338 (1996), the entirety of which is herein incorporated by reference). As used herein, reference to the U03 linkage group of Glycine max refers to the linkage group that corresponds to U03 linkage group from the genetic map of Glycine max (Mansur et al., Crop Science. 36: 1327-1336 (1996); Cregan et al., Crop Science 39:1464-1490 (1999), and Soybase, Agricultural Research Service, United States Department of Agriculture (http://129.186.26.940/ and USDA-Agricultural Research Service:http://www.ars.usda.gov/)).

An allele of a quantitative trait locus can, of course, comprise multiple genes or other genetic factors even within a contiguous genomic region or linkage group. As used herein, an allele of a quantitative trait locus can therefore encompasses more than one gene or other genetic factor where each individual gene or genetic component is also capable of exhibiting allelic variation and where each gene or genetic factor also has a phenotypic effect on the quantitative trait in question. In an embodiment of the present invention the allele of a quantitative trait locus comprises one or more genes or other genetic factors that are also capable of exhibiting allelic variation. The use of the term “an allele of a quantitative trait locus” is thus not intended to exclude a quantitative trait locus that comprises more than one gene or other genetic factor. As used herein, an allele is one of several alternative forms of a gene occupying a given locus on a chromosome. When all the alleles present at a given locus on a chromosome are the same that plant is homozygous at that locus. If the alleles present at a given locus on a chromosome differ that plant is heterozygous at that locus.

In another embodiment, a yellow seed coat Glycine max plant of the present invention has an allele of an enhanced yield quantitative trait locus that is genetically linked to the marker nucleic acid molecule selected from the group comprising Satt187, Sat_{—}212, Sat_{—}215, Sy50, SCNB190, SCNB188, SAHH, SCNB187, XET1, Sy36, Satt315, and chalcone synthase gene cluster DNA sequences.

In an embodiment, a yellow seed coat Glycine max plant of the present invention exhibits an enhanced yield as measured by dry seed weight. The enhanced yield is measured as dry seed weight at about 13% moisture content in comparison to a Glycine max plant of a similar genetic background grown under similar conditions, but whose genetic makeup lacks the alleles of a quantitative trait locus associated with enhanced yield introgressed from the Glycine max PI290136 plant, where the alleles of a quantitative trait locus are also located on linkage group U03 of a Glycine max PI290136 plant. In an embodiment the enhanced yield results in a greater than 2% increase in average dry seed weight. In a preferred embodiment the enhanced yield results in a greater than 4% increase in average dry seed weight. In a more preferred embodiment the enhanced yield results in a greater than 5% increase in average dry seed weight. In an even more preferred embodiment the enhanced yield results in a greater than 10% increase in average dry seed weight. In an even more preferred embodiment the enhanced yield results in a greater than 12% increase in average dry seed weight. In a particularly preferred embodiment the enhanced yield results in a greater than 14% or greater than 18% increase in average dry seed weight.

Many agronomic traits can affect yield. These include, without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, height, resistance to lodging, percent seed germination, seedling vigor, and juvenile traits. In an embodiment, a Glycine max plant of the present invention exhibits an enhanced trait that is a component of yield.

Heterogeneity can exist in any Glycine max accession and specifically that heterogeneity may exist in the exotic Glycine max PI290136. It is further understood that in light of the current disclosure, Glycine max PI290136 having an allele of a quantitative trait locus located on linkage group U03 and associated with enhanced yield in elite Glycine max plant can be screened for using one or more the techniques described herein or known in the art. In a preferred embodiment single seed selection from the segregating progeny of PI290136 is used in a backcross with an elite Glycine max lines such as H5050 and CX445. The presence or absence of alleles from Glycine max PI290136 can, for example, be determined in the BC₂F₄ generation.

The present invention also provides a yellow seed coat Glycine max plant, which exhibits an enhanced yield compared to a first parent, the Glycine max plant having a genome homozygous or heterozygous with respect to a genetic allele that is native to a second parent selected from the group consisting of Glycine max PI290136 and progeny thereof and non-native to a first parent, where the first parent is an elite Glycine max plant.

Moreover, the present invention also provides a elite yellow seed coat Glycine max plant comprising an allele of a quantitative trait locus derived from an exotic Glycine plant, wherein the quantitative trait locus is also located on linkage group U03 of Glycine max PI290136.

Furthermore, the present invention provides a method for the production of an elite Glycine max plant having enhanced yield comprising: (A) crossing a Glycine max PI290136 plant or progeny thereof with an elite Glycine max plant to produce a segregating population; (B) screening the segregating population for a member having an allele derived from Glycine max PI290136 plant or progeny thereof that mapped to linkage group U03 of the Glycine max PI290136 plant or progeny thereof, where the allele is associated with the enhanced yield in the Glycine max plant; and (C) selecting the member for further crossing and selection, wherein the member selected has the allele derived from Glycine max PI290136 plant or progeny thereof that mapped to linkage group U03 and has a yellow seed coat.

Plants of the present invention can be part of or generated from a breeding program. The choice of breeding method depends on the mode of plant reproduction, the heritability of the trait(s) being improved, and the type of cultivar used commercially (e.g., F₁ hybrid cultivar, pureline cultivar, etc). A cultivar is a race or variety of a plant that has been created or selected intentionally and maintained through cultivation.

Selected, non-limiting approaches, for breeding the plants of the present invention are set forth below. A breeding program can be enhanced using marker assisted selection of the progeny of any cross. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability etc. will generally dictate the choice.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred embodiment a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Breeding lines can be tested and compared to appropriate standards in environments representative of the commercial target area(s) for two or more generations. The best lines are candidates for new commercial cultivars; those still deficient in traits may be used as parents to produce new populations for further selection.

One method of identifying a superior plant is to observe its performance relative to other experimental plants and to a widely grown standard cultivar. If a single observation is inconclusive, replicated observations can provide a better estimate of its genetic worth. A breeder can select and cross two or more parental lines, followed by repeated selfing and selection, producing many new genetic combinations.

The development of new soybean cultivars requires the development and selection of soybean varieties, the crossing of these varieties and selection of superior hybrid crosses. The hybrid seed can be produced by manual crosses between selected male-fertile parents or by using male sterility systems. Hybrids are selected for certain single gene traits such as pod color, flower color, seed yield, pubescence color or herbicide resistance which indicate that the seed is truly a hybrid. Additional data on parental lines, as well as the phenotype of the hybrid, influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Pedigree breeding is used commonly for the improvement of self-pollinating crops. Two parents who possess favorable, complementary traits are crossed to produce an F₁. An F₂ population is produced by selfing one or several Fl's. Selection of the best individuals in the best families is selected. Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Backcross breeding has been used to transfer genes for a simply inherited, highly heritable trait into a desirable homozygous cultivar or inbred line, which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In a multiple-seed procedure, soybean breeders commonly harvest one or more pods from each plant in a population and thresh them together to form a bulk. Part of the bulk is used to plant the next generation and part is put in reserve. The procedure has been referred to as modified single-seed descent or the pod-bulk technique.

The multiple-seed procedure has been used to save labor at harvest. It is considerably faster to thresh pods with a machine than to remove one seed from each by hand for the single-seed procedure. The multiple-seed procedure also makes it possible to plant the same number of seed of a population each generation of inbreeding.

Descriptions of other breeding methods that are commonly used for different traits and crops can be found in one of several reference books (e.g. Fehr, Principles of Cultivar Development Vol. 1, pp. 2-3 (1987)), the entirety of which is herein incorporated by reference).

The present invention also provides for parts of the plants of the present invention. Plant parts, without limitation, include seed, endosperm, ovule and pollen. In a particularly preferred embodiment of the present invention, the plant part is a seed.

Moreover, the present invention also provides for a container having more than 40,000 Glycine max seeds where over 40% of the seeds are from plants of the present invention. The present invention also provides for a container having more than 80,000 Glycine max seeds where over 40% of the seeds are from plants of the present invention.

In a preferred embodiment, the present invention also provides for a container having more than 40,000 Glycine max seeds where over 60% of the seeds are from plants of the present invention. In another preferred embodiment, the present invention also provides for a container having more than 80,000 Glycine max seeds where over 60% of the seeds are from plants of the present invention. In an even more preferred embodiment, the present invention also provides for a container having more than 40,000 Glycine max seeds where over 80% of the seeds are from plants of the present invention. In another even more preferred embodiment, the present invention also provides for a container having more than 80,000 Glycine max seeds where over 80% of the seeds are from plants of the present invention. In a further even more preferred embodiment, the present invention also provides for a container having more than 40,000 Glycine max seeds where over 90% of the seeds are from plants of the present invention. In another preferred embodiment, the present invention also provides for a container having more than 80,000 Glycine max seeds where over 90% of the seeds are from plants of the present invention.

Moreover, the present invention also provides for a container having more than 25 lbs. of Glycine max seeds where over 40% of the seeds are from plants of the present invention. The present invention also provides for a container having more than 40 lbs. of Glycine max seeds where over 40% of the seeds are from plants of the present invention. In a preferred embodiment, the present invention also provides for a container having more than 25 lbs. of Glycine max seeds where over 60% of the seeds are from plants of the present invention. In another preferred embodiment, the present invention also provides for a container having more than 40 lbs. of Glycine max seeds where over 60% of the seeds are from plants of the present invention. In an even more preferred embodiment, the present invention also provides for a container having more than 25 lbs. of Glycine max seeds where over 80% of the seeds are from plants of the present invention. In another even more preferred embodiment, the present invention also provides for a container having more than 40 lbs. of Glycine max seeds where over 80% of the seeds are from plants of the present invention. In a further even more preferred embodiment, the present invention also provides for a container having more than 25 lbs. of Glycine max seeds where over 90% of the seeds are from plants of the present invention. In another preferred embodiment, the present invention also provides for a container having more than 40 lbs. of Glycine max seeds where over 90% of the seeds are from plants of the present invention.

Plants or parts thereof of the present invention may be grown in culture and regenerated. Methods for the regeneration of Glycine max plants from various tissue types and methods for the tissue culture of Glycine max are known in the art (See, for example, Widholm et al., In Vitro Selection and Culture-induced Variation in Soybean, In Soybean: Genetics, Molecular Biology and Biotechnology, Eds. Verma and Shoemaker, CAB International, Wallingford, Oxon, England (1996). Regeneration techniques for plants such as Glycine max can use as the starting material a variety of tissue or cell types. With Glycine max in particular, regeneration processes have been developed that begin with certain differentiated tissue types such as meristems, Cartha et al., Can. J. Bot. 59:1671-1679 (1981), hypocotyl sections, Cameya et al., Plant Science Letters 21: 289-294 (1981), and stem node segments, Saka et al., Plant Science Letters, 19: 193-201 (1980); Cheng et al., Plant Science Letters, 19: 91-99 (1980). Regeneration of whole sexually mature Glycine max plants from somatic embryos generated from explants of immature Glycine max embryos has been reported (Ranch et al., In Vitro Cellular & Developmental Biology 21: 653-658 (1985). Regeneration of mature Glycine max plants from tissue culture by organogenesis and embryogenesis has also been reported (Barwale et al., Planta 167: 473-481 (1986); Wright et al., Plant Cell Reports 5: 150-154 (1986).

The present invention also provides a yellow seed coat Glycine max plant selected for by screening for an enhanced yield in the Glycine max plant, the selection comprising interrogating genomic DNA for the presence of a marker molecule that is genetically linked to an allele of a quantitative trait locus associated with enhanced yield in the Glycine max plant, where the allele of a quantitative trait locus is also located on linkage group U03 of a Glycine max PI290136 plant.

It is further understood, that the present invention provides bacterial, viral, microbial, insect, mammalian and plant cells comprising the agents of the present invention.

Nucleic acid molecules or fragments thereof are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., In: Molecular Cloning, A Laboratory Manual, 2nd Edition, Cold Spring Harbor Press, Cold Spring Harbor, N.Y. (1989)), and by Haymes et al., In: Nucleic Acid Hybridization, A Practical Approach, IRL Press, Washington, D.C. (1985), the entirety of which is herein incorporated by reference. Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

As used herein, a substantially homologous sequence is a nucleic acid sequence that will specifically hybridize to the complement of the nucleic acid sequence to which it is being compared under high stringency conditions. The nucleic-acid probes and primers of the present invention can hybridize under stringent conditions to a target DNA sequence. The term “stringent hybridization conditions” is defined as conditions under which a probe or primer hybridizes specifically with a target sequence(s) and not with non-target sequences, as can be determined empirically. The term “stringent conditions” is functionally defined with regard to the hybridization of a nucleic-acid probe to a target nucleic acid (i.e., to a particular nucleic-acid sequence of interest) by the specific hybridization procedure discussed in Sambrook et al., 1989, at 9.52-9.55. See also, Sambrook et al., 1989 at 9.47-9.52, 9.56-9.58; Kanehisa, Nucl. Acids Res. 12:203-213, 1984; and Wetmur and Davidson, J. Mol. Biol. 31:349-370, 1968. Appropriate stringency conditions that promote DNA hybridization are, for example, 6.0× sodium chloride/sodium citrate (SSC) at about 45° C., followed by a wash of 2.0× SSC at 50° C., are known to those skilled in the art or can be found in Current Protocols in Molecular Biology, John Wiley & Sons, N.Y., 1989, 6.3.1-6.3.6. For example, the salt concentration in the wash step can be selected from a low stringency of about 2.0× SSC at 50° C. to a high stringency of about 0.2× SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

For example, hybridization using DNA or RNA probes or primers can be performed at 65° C. in 6× SSC, 0.5% SDS, 5× Denhardt's, 100 μg/mL nonspecific DNA (e.g., sonicated salmon sperm DNA) with washing at 0.5× SSC, 0.5% SDS at 65° C., for high stringency.

It is contemplated that lower stringency hybridization conditions such as lower hybridization and/or washing temperatures can be used to identify related sequences having a lower degree of sequence similarity if specificity of binding of the probe or primer to target sequence(s) is preserved. Accordingly, the nucleotide sequences of the present invention can be used for their ability to selectively form duplex molecules with complementary stretches of DNA fragments. Detection of DNA segments via hybridization is well-known to those of skill in the art, and thus depending on the application envisioned, one will desire to employ varying hybridization conditions to achieve varying degrees of selectivity of probe towards target sequence and the method of choice will depend on the desired results.

As used herein, an agent, be it a naturally occurring molecule or otherwise may be “substantially purified”, if desired, referring to a molecule separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

The agents of the present invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic, and thus involve the capacity of the agent to mediate a chemical reaction or response.

The agents of the present invention may also be recombinant. As used herein, the term recombinant means any agent (e.g. DNA, peptide etc.), that is, or results, however indirect, from human manipulation of a nucleic acid molecule.

The agents of the present invention may be labeled with reagents that facilitate detection of the agent (e.g. fluorescent labels (Prober et al., Science 238:336-340 (1987); Albarella et al., European Patent 144914), chemical labels (Sheldon et al., U.S. Pat. No. 4,582,789; Albarella et al., U.S. Pat. No. 4,563,417), modified bases (Miyoshi et al., European Patent 119448), all of which are herein incorporated by reference in their entirety).

In a preferred embodiment, a nucleic acid of the present invention will specifically hybridize to one or more of the nucleic acid molecules set forth in SEQ ID NO: 19 through SEQ ID NO:37 or complements thereof or fragments of either under moderately stringent conditions, for example at about 2.0× SSC and about 65° C. In a particularly preferred embodiment, a nucleic acid of the present invention will specifically hybridize to one or more of the nucleic acid molecules set forth in SEQ ID NO: 19 through SEQ ID NO:37 or complements or fragments of either under high stringency conditions. In one aspect of the present invention, a preferred marker nucleic acid molecule of the present invention has the nucleic acid sequence set forth in SEQ ID NO: 19 through SEQ ID NO:37 or complements thereof or fragments of either. In another aspect of the present invention, a preferred marker nucleic acid molecule of the present invention shares between 80% and 100% or 90% and 100% sequence identity with the nucleic acid sequence set forth in SEQ ID NO: 19 through SEQ ID NO:37 or complement thereof or fragments of either. In a further aspect of the present invention, a preferred marker nucleic acid molecule of the present invention shares between 95% and 100% sequence identity with the sequence set forth in SEQ ID NO: 19 through SEQ ID NO:37 or complement thereof or fragments of either. In a more preferred aspect of the present invention, a preferred marker nucleic acid molecule of the present invention shares between 98% and 100% sequence identity with the nucleic acid sequence set forth in SEQ ID NO: 19 through SEQ ID NO:37 or complement thereof or fragments of either.

Additional genetic markers can be used to select plants with an allele of a quantitative trait locus associated with enhanced yield in Glycine max of the present invention. Examples of public marker databases include, for example: Soybase, an Agricultural Research Service, United States Department of Agriculture (http://129.186.26.940/ and USDA-Agricultural Research Service: http://www.ars.usda.gov/).

A preferred group of markers is selected from the group consisting of a marker nucleic acid molecule that specifically hybridizes to Satt187, Sat_{—}212, Sat_{—}215, Sy50,SCNB190, SCNB188, SAHH, SCNB187, XET1, Sy36, and Satt315, chalcone synthase gene cluster sequences or their complement. In a preferred embodiment, the genetic marker of the present invention is a SSR.

Polymorphisms may also be found using a DNA fingerprinting technique called amplified fragment length polymorphism (AFLP), which is based on the selective PCR amplification of restriction fragments from a total digest of genomic DNA to profile that DNA (Vos et al., Nucleic Acids Res. 23:4407-4414 (1995), the entirety of which is herein incorporated by reference). This method allows for the specific co-amplification of high numbers of restriction fragments, which can be visualized by PCR without knowledge of the nucleic acid sequence.

AFLP employs basically three steps. Initially, a sample of genomic DNA is cut with restriction enzymes and oligonucleotide adapters are ligated to the restriction fragments of the DNA. The restriction fragments are then amplified using PCR by using the adapter and restriction sequence as target sites for primer annealing. The selective amplification is achieved by the use of primers that extend into the restriction fragments, amplifying only those fragments in which the primer extensions match the nucleotide flanking the restriction sites. These amplified fragments are then visualized on a denaturing polyacrylamide gel.

AFLP analysis has been performed on Salix (Beismann et al., Mol. Ecol. 6:989-993 (1997), the entirety of which is herein incorporated by reference), Acinetobacter (Janssen et al., Int. J. Syst. Bacteriol. 47:1179-1187 (1997), the entirety of which is herein incorporated by reference), Aeromonas popoffi (Huys et al., Int. J. Syst. Bacteriol. 47:1165-1171 (1997), the entirety of which is herein incorporated by reference), rice (McCouch et al., Plant Mol. Biol. 35:89-99 (1997), the entirety of which is herein incorporated by reference), Nandi et al., Mol. Gen. Genet. 255:1-8 (1997), the entirety of which is herein incorporated by reference; Cho et al., Genome 39:373-378 (1996), the entirety of which is herein incorporated by reference), barley (Hordeum vulgare)(Simons et al., Genomics 44:61-70 (1997), the entirety of which is herein incorporated by reference; Waugh et al., Mol. Gen. Genet. 255:311-321 (1997), the entirety of which is herein incorporated by reference; Qi et al., Mol. Gen Genet. 254:330-336 (1997), the entirety of which is herein incorporated by reference; Becker et al., Mol. Gen. Genet. 249:65-73 (1995), the entirety of which is herein incorporated by reference), potato (Van der Voort et al., Mol. Gen. Genet. 255:438-447 (1997), the entirety of which is herein incorporated by reference; Meksem et al., Mol. Gen. Genet. 249:74-81 (1995), the entirety of which is herein incorporated by reference), Phytophthora infestans (Van der Lee et al., Fungal Genet. Biol. 21:278-291 (1997), the entirety of which is herein incorporated by reference), Bacillus anthracis (Keim et al., J. Bacteriol. 179:818-824 (1997), the entirety of which is herein incorporated by reference), Astragalus cremnophylax (Travis et al., Mol. Ecol. 5:735-745 (1996), the entirety of which is herein incorporated by reference), Arabidopsis thaliana (Cnops et al., Mol. Gen. Genet. 253:32-41 (1996), the entirety of which is herein incorporated by reference), Escherichia coli (Lin et al., Nucleic Acids Res. 24:3649-3650 (1996), the entirety of which is herein incorporated by reference), Aeromonas (Huys et al., Int. J. Syst. Bacteriol. 46:572-580 (1996), the entirety of which is herein incorporated by reference), nematode (Folkertsma et al., Mol. Plant Microbe Interact. 9:47-54 (1996), the entirety of which is herein incorporated by reference), tomato (Thomas et al., Plant J. 8:785-794 (1995), the entirety of which is herein incorporated by reference), and human (Latorra et al., PCR Methods Appl. 3:351-358 (1994), the entirety of which is herein incorporated by reference). AFLP analysis has also been used for fingerprinting mRNA (Money et al., Nucleic Acids Res. 24:2616-2617 (1996), the entirety of which is herein incorporated by reference; Bachem et al., Plant J. 9:745-753 (1996), the entirety of which is herein incorporated by reference). It is understood that one or more of the nucleic acids of the present invention, can be utilized as markers or probes to detect polymorphisms by AFLP analysis or for fingerprinting RNA.

In a preferred embodiment, a marker molecule is detected by DNA amplification using a forward and a reverse primer capable of detecting a marker molecule of the present invention. In a particularly preferred embodiment, a marker molecule is detected by AFLP amplification.

Microsatellite (SSR) markers have been used to distinguish the genotype of soybean cultivars and elite breeding lines. These methods have been developed for soybean and are well known in the field of molecular plant breeding (Rongwen, Theor. Appl. Gen. 90:43-48 (1995); Akkaya, Crop Sci. 35:1439-1445 (1995); Mansur, Crop Sci. 36:1327-1336 (1996); Diwan, Theor. Appl. Gen. 95:723-733 (1997); Simple sequence repeat DNA marker analysis, in “DNA markers: Protocols, applications, and overviews: (1997) 173-185, Cregan, et al., eds., Wiley-Liss NY; all of which is herein incorporated by reference in its' entirely. In a particularly preferred embodiment, a marker molecule is detected by SSR techniques. It is understood that SSR and AFLP primers can hybridize to a combination of plant DNA and adapter DNA (e.g. EcoRI adapter or MseI adapter, Vos et al., Nucleic Acids Res. 23:4407-4414 (1995)).

Genetic markers of the present invention include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

Additional markers, such as microsatellite markers (SSR), AFLP markers, RFLP markers, RAPD markers, phenotypic markers, SNPs, isozyme markers, microarray transcription profiles that are genetically linked to or correlated with alleles of a QTL of the present invention can be utilized (Walton, Seed World 22-29 (July, 1993), the entirety of which is herein incorporated by reference; Burow and Blake, Molecular Dissection of Complex Traits, 13-29, Eds. Paterson, CRC Press, New York (1988), the entirety of which is herein incorporated by reference). Methods to isolate such markers are known in the art. For example, locus-specific microsatellite markers (SSR) can be obtained by screening a genomic library for microsatellite repeats, sequencing of “positive” clones, designing primers which flank the repeats, and amplifying genomic DNA with these primers. The size of the resulting amplification products can vary by integral numbers of the basic repeat unit. To detect a polymorphism, PCR products can be radiolabeled, separated on denaturing polyacrylamide gels, and detected by autoradiography. Fragments with size differences >4 bp can also be resolved on agarose gels, thus avoiding radioactivity.

Other microsatellite markers may be utilized. Amplification of simple tandem repeats, mainly of the [CA]ₙ type were reported by Litt and Luty, Amer. J. Human Genet. 44:397-401 (1989), the entirety of which is herein incorporated by reference; Smeets et al., Human Genet. 83:245-251 (1989), the entirety of which is herein incorporated by reference; Tautz, Nucleic Acids Res. 17:6463-6472 (1989), the entirety of which is herein incorporated by reference; Weber and May, Am. J. Hum. Genet. 44:388-396 (1989), the entirety of which is herein incorporated by reference. Weber Genomics 7:524-530 (1990), the entirety of which is herein incorporated by reference, reported that the level of polymorphism detected by PCR-amplified [CA]ₙ type microsatellites depends on the number of the “perfect” (i.e., uninterrupted), tandemly repeated motifs. Below a certain threshold (i.e., 12 CA-repeats), the microsatellites were reported to be primarily monomorphic. Above this threshold, however, the probability of polymorphism increases with microsatellite length. Consequently, long, perfect arrays of microsatellites are preferred for the generation of markers, i.e., for the design and synthesis of flanking primers.

Suitable primers can be deduced from DNA databases (e.g., Akkaya et al., Genetics. 132:1131-1139 (1992), the entirety of which is herein incorporated by reference). Alternatively, size-selected genomic libraries (200 to 500 bp) can be constructed by, for example, using the following steps: (1) isolation of genomic DNA; (2) digestion with one or more 4 base-specific restriction enzymes; (3) size-selection of restriction fragments by agarose gel electrophoresis, excision and purification of the desire size fraction; (4) ligation of the DNA into a suitable vector and transformation into a suitable E. coli strain; (5) screening for the presence of microsatellites by colony or plaque hybridization with a labeled probe; (6) isolation of positive clones and sequencing of the inserts; and (7) design of suitable primers flanking the microsatellite repeat.

Establishing libraries with small, size-selected inserts can be advantageous for microsatellite isolation for two reasons: (1) long microsatellites are often unstable in E. coli, and (2) positive clones can be sequenced without subcloning. A number of approaches have been reported for the enrichment of microsatellites in genomic libraries. Such enrichment procedures are particularly useful if libraries are screened with comparatively rare tri- and tetranucleotide repeat motifs. One such approach has been described by Ostrander et al., Proc. Natl. Acad. Sci. (U.S.A). 89:3419-3423 (1992), the entirety of which is herein incorporated by reference, who reported the generation of a small-insert phagemid library in an E. coli strain deficient in UTPase (d8t) and uracil-N-glycosylase (ung) genes. In the absence of UTPase and uracil-N-glycosylase, dUTP can compete with dTTP for the incorporation into DNA. Single-stranded phagemid DNA isolated from such a library, can be primed with [CA]ₙ and [TG]ₙ primers for second strand synthesis, and the products used to transform a wild-type E. coli strain. Since under these conditions there will be selection against single-stranded, uracil-containing DNA molecules, the resulting library will consist of primer-extended, double-stranded products and an about 50-fold enrichment in CA-repeats.

Other reported enrichment strategies rely on hybridization selection of simple sequence repeats prior to cloning (Karagyozov et al., Nucleic Acids Res. 21:3911-3912 (1993), the entirety of which is herein incorporated by reference; Armour et al., Hum. Mol. Gen. 3:599-605 (1994), the entirety of which is herein incorporated by reference; Kijas et al., Genome 38:349-355 (1994), the entirety of which is herein incorporated by reference; Kandpal et al., Proc. Natl. Acad. Sci. (U.S.A.) 91:88-92 (1994), the entirety of which is herein incorporated by reference; Edwards et al., Am. J. Hum. Genet. 49:746-756 (1991), the entirety of which is herein incorporated by reference). Hybridization selection, can for example, involve the following steps: (1) genomic DNA is fragmented, either by sonication, or by digestion with a restriction enzyme; (2) genomic DNA fragments are ligated to adapters that allow a “whole genome PCR” at this or a later stage of the procedure; (3) genomic DNA fragments are amplified, denatured and hybridized with single-stranded microsatellite sequences bound to a nylon membrane; (4) after washing off unbound DNA, hybridizing fragments enriched for microsatellites are eluted from the membrane by boiling or alkali treatment, reamplified using adapter-complementary primers, and digested with a restriction enzyme to remove the adapters; and (5) DNA fragments are ligated into a suitable vector and transformed into a suitable E. coli strain. Microsatellite can be found in up to 50-70% of the clones obtained from these procedures (Armour et al., Hum. Mol. Gen. 3:599-605 (1994), the entirety of which is herein incorporated by reference; Edwards et al., Am. J. Hum. Genet. 49:746-756 (1991), the entirety of which is herein incorporated by reference.

An alternative hybridization selection strategy was reported by Kij as et al, Genome 38:599-605 (1994), the entirety of which is herein incorporated by reference, which replaced the nylon membrane with biotinylated, microsatellite-complementary oligonucleotides attached to streptavidin-coated magnetic particles. Microsatellite-containing DNA fragments are selectively bound to the magnetic beads, reamplified, restriction-digested and cloned.

It is further understood that other additional markers on linkage group U03 may be utilized (Morgante et al., Genome 37:763-769 (1994), the entirety of which is herein incorporated by reference in its entirety). PCR-amplified microsatellites can be used, because they are locus-specific, codominant, occur in large numbers and allow the unambiguous identification of alleles. Standard PCR-amplified microsatellites protocols use radioisotopes and denaturing polyacrylamide gels to detect amplified microsatellites. In many situations, however, allele sizes are sufficiently different to be resolved on high percentage agarose gels in combination with ethidium bromide staining (Bell and Ecker, Genomics 19:137-144 (1994), the entirety of which is herein incorporated by reference; Becker and Heun, Genome 38:991-998 (1995), the entirety of which is herein incorporated by reference; Huttel, Ph.D. Thesis, University of Frankfurt, Germany (1996), the entirety of which is herein incorporated by reference). High resolution without applying radioactivity is also provided by nondenaturing polyacrylamide gels in combination with either ethidium bromide (Scrimshaw, Biotechniques 13:2189 (1992), the entirety of which is herein incorporated by reference) or silver straining (Klinkicht and Tautz, Molecular Ecology 1: 133-134 (1992), the entirety of which is herein incorporated by reference; Neilan et al., Biotechniques 17:708-712 (1994), the entirety of which is herein incorporated by reference). An alternative of PCR-amplified microsatelllites typing involves the use of fluorescent primers in combination with a semi-automated DNA sequencer (Schwengel et al., Genomics 22:46-54 (1994), the entirety of which is herein incorporated by reference). Fluorescent PCR products can be detected by real-time laser scanning during gel electrophoresis. An advantage of this technology is that different amplification reactions as well as a size marker (each labeled with a different fluorophore) can be combined into one lane during electrophoresis. Multiplex analysis of up to 24 different microsatellite loci per lane has been reported (Schwengel et al., Genomics 22:46-54 (1994)).

The detection of polymorphic sites in a sample of DNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means.

The most preferred method of achieving such amplification employs the polymerase chain reaction (“PCR”) (Mullis et al., Cold Spring Harbor Symp. Quant. Biol. 51:263-273 (1986); Erlich et al., European Patent Appln. 50,424; European Patent Appln. 84,796, European Patent Application 258,017, European Patent Appln. 237,362; Mullis, European Patent Appln. 201,184; Mullis et al., U.S. Pat. No. 4,683,202; Erlich, U.S. Pat. No. 4,582,788; and Saiki et al., U.S. Pat. No. 4,683,194), using primer pairs that are capable of hybridizing to the proximal sequences that define a polymorphism in its double-stranded form.

In lieu of PCR, alternative methods, such as the “Ligase Chain Reaction” (“LCR”) may be used (Barany, Proc. Natl. Acad. Sci. (U.S.A.) 88:189-193 (1991), the entirety of which is herein incorporated by reference). LCR uses two pairs of oligonucleotide probes to exponentially amplify a specific target. The sequences of each pair of oligonucleotides is selected to permit the pair to hybridize to abutting sequences of the same strand of the target. Such hybridization forms a substrate for a template-dependent ligase. As with PCR, the resulting products thus serve as a template in subsequent cycles and an exponential amplification of the desired sequence is obtained.

LCR can be performed with oligonucleotides having the proximal and distal sequences of the same strand of a polymorphic site. In one embodiment, either oligonucleotide will be designed to include the actual polymorphic site of the polymorphism. In such an embodiment, the reaction conditions are selected such that the oligonucleotides can be ligated together only if the target molecule either contains or lacks the specific nucleotide that is complementary to the polymorphic site present on the oligonucleotide. Alternatively, the oligonucleotides may be selected such that they do not include the polymorphic site (see, Segev, PCT Application WO 90/01069, the entirety of which is herein incorporated by reference).

The “Oligonucleotide Ligation Assay” (“OLA”) may alternatively be employed (Landegren et al., Science 241: 1077-1080 (1988), the entirety of which is herein incorporated by reference). The OLA protocol uses two oligonucleotides that are designed to be capable of hybridizing to abutting sequences of a single strand of a target. OLA, like LCR, is particularly suited for the detection of point mutations. Unlike LCR, however, OLA results in “linear” rather than exponential amplification of the target sequence.

Nickerson et al. have described a nucleic acid detection assay that combines attributes of PCR and OLA (Nickerson et al., Proc. Natl. Acad. Sci. (U.S.A.) 87:8923-8927 (1990), the entirety of which is herein incorporated by reference). In this method, PCR is used to achieve the exponential amplification of target DNA, which is then detected using OLA. In addition to requiring multiple, and separate, processing steps, one problem associated with such combinations is that they inherit all of the problems associated with PCR and OLA.

Schemes based on ligation of two (or more) oligonucleotides in the presence of a nucleic acid having the sequence of the resulting “di-oligonucleotide”, thereby amplifying the di-oligonucleotide, are also known (Wu et al., Genomics 4:560-569 (1989), the entirety of which is herein incorporated by reference), and may be readily adapted to the purposes of the present invention.

Other known nucleic acid amplification procedures, such as allele-specific oligomers, branched DNA technology, transcription-based amplification systems, or isothermal amplification methods may also be used to amplify and analyze such polymorphisms (Malek et al., U.S. Pat. No. 5,130,238; Davey et al., European Patent Application 329,822; Schuster et al., U.S. Pat. No. 5,169,766; Miller et al., PCT Patent Application WO 89/06700; Kwoh, et al., Proc. Natl. Acad. Sci. (U.S.A.) 86:1173-1177 (1989); Gingeras et al., PCT Patent Application WO 88/10315; Walker et al., Proc. Natl. Acad. Sci. (U.S.A.) 89:392-396 (1992), all of which are herein incorporated by reference in their entirety).

Polymorphisms can also be identified by Single Strand Conformation Polymorphism (SSCP) analysis. SSCP is a method capable of identifying most sequence variations in a single strand of DNA, typically between 150 and 250 nucleotides in length (Elles, Methods in Molecular Medicine: Molecular Diagnosis of Genetic Diseases, Humana Press (1996), the entirety of which is herein incorporated by reference; Orita et al., Genomics 5: 874-879 (1989), the entirety of which is herein incorporated by reference). Under denaturing conditions a single strand of DNA will adopt a conformation that is uniquely dependent on its sequence conformation. This conformation usually will be different, even if only a single base is changed. Most conformations have been reported to alter the physical configuration or size sufficiently to be detectable by electrophoresis. A number of protocols have been described for SSCP including, but not limited to, Lee et al., Anal. Biochem. 205: 289-293 (1992), the entirety of which is herein incorporated by reference; Suzuki et al., Anal. Biochem. 192: 82-84 (1991), the entirety of which is herein incorporated by reference; Lo et al., Nucleic Acids Research 20: 1005-1009 (1992), the entirety of which is herein incorporated by reference; Sarkar et al., Genomics 13:441-443 (1992), the entirety of which is herein incorporated by reference. It is understood that one or more of the nucleic acids of the present invention, can be utilized as markers or probes to detect polymorphisms by SSCP analysis.

Polymorphisms may also be found using random amplified polymorphic DNA (RAPD) (Williams et al., Nucl. Acids Res. 18: 6531-6535 (1990), the entirety of which is herein incorporated by reference) and cleaveable amplified polymorphic sequences (CAPS) (Lyamichev et al., Science 260: 778-783 (1993), the entirety of which is herein incorporated by reference). It is understood that one or more of the nucleic acid molecules of the present invention, can be utilized as markers or probes to detect polymorphisms by RAPD or CAPS analysis.

The identification of a polymorphism can be determined in a variety of ways. By correlating the presence or absence of it in a plant with the presence or absence of a phenotype, it is possible to predict the phenotype of that plant. If a polymorphism creates or destroys a restriction endonuclease cleavage site, or if it results in the loss or insertion of DNA (e.g., a variable nucleotide tandem repeat (VNTR) polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms” (“RFLPs”). RFLPs have been widely used in human and plant genetic analyses (Glassberg, UK Patent Application 2135774; Skolnick et al., Cytogen. Cell Genet. 32:58-67 (1982); Botstein et al., Ann. J. Hum. Genet. 32:314-331 (1980); Fischer et al. (PCT Application WO90/13668); Uhlen, PCT Application WO90/11369).

A central attribute of “single nucleotide polymorphisms,” or “SNPs” is that the site of the polymorphism is at a single nucleotide. SNPs have certain reported advantages over RFLPs and VNTRs. First, SNPs are more stable than other classes of polymorphisms. Their spontaneous mutation rate is approximately 10⁻⁹ (Kornberg, DNA Replication, W. H. Freeman & Co., San Francisco, 1980), approximately 1,000 times less frequent than VNTRs (U.S. Pat. No. 5,679,524, the entirety of which is herein incorporated by reference). Second, SNPs occur at greater frequency, and with greater uniformity than RFLPs and VNTRs. As SNPs result from sequence variation, new polymorphisms can be identified by sequencing random genomic or cDNA molecules. SNPs can also result from deletions, point mutations and insertions. Any single base alteration, whatever the cause, can be a SNP. The greater frequency of SNPs means that they can be more readily identified than the other classes of polymorphisms.

SNPs can be characterized using any of a variety of methods. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism or by other biochemical interpretation. SNPs can sequenced by a number of methods. Two basic methods may be used for DNA sequencing, the chain termination method of Sanger et al., Proc. Natl. Acad. Sci. (U.S.A.) 74: 5463-5467 (1977), the entirety of which is herein incorporated by reference and the chemical degradation method of Maxam and Gilbert, Proc. Nat. Acad. Sci. (U.S.A.) 74: 560-564 (1977), the entirety of which is herein incorporated by reference. Automation and advances in technology such as the replacement of radioisotopes with fluorescence-based sequencing have reduced the effort required to sequence DNA (Craxton, Methods, 2: 20-26 (1991), the entirety of which is herein incorporated by reference; Ju et al., Proc. Natl. Acad. Sci. (U.S.A.) 92: 4347-4351 (1995), the entirety of which is herein incorporated by reference; Tabor and Richardson, Proc. Natl. Acad. Sci. (U.S.A.) 92: 6339-6343 (1995), the entirety of which is herein incorporated by reference). Automated sequencers are available from, for example, Pharmacia Biotech, Inc., Piscataway, N.J. (Pharmacia ALF), LI-COR, Inc., Lincoln, Ner. (LI-COR 4,000) and Millipore, Bedford, Mass. (Millipore BaseStation).

In addition, advances in capillary gel electrophoresis have also reduced the effort required to sequence DNA and such advances provide a rapid high resolution approach for sequencing DNA samples (Swerdlow and Gesteland, Nucleic Acids Res. 18:1415-1419 (1990); Smith, Nature 349:812-813 (1991); Luckey et al., Methods Enzymol. 218:154-172 (1993); Lu et al., J. Chromatog. A. 680:497-501 (1994); Carson et al., Anal. Chem. 65:3219-3226 (1993); Huang et al., Anal. Chem. 64:2149-2154 (1992); Kheterpal et al., Electrophoresis 17:1852-1859 (1996); Quesada and Zhang, Electrophoresis 17:1841-1851 (1996); Baba, Yakugaku Zasshi 117:265-281 (1997), Marino, Appl. Theor. Electrophor. 5:1-5 (1995); all of which are herein incorporated by reference in their entirety).

A microarray-based method for high-throughput monitoring of plant gene expression can be utilized as a genetic marker system. This ‘chip’-based approach involves using microarrays of nucleic acid molecules as gene-specific hybridization targets to quantitatively or qualitatively measure expression of plant genes (Schena et al., Science 270:467-470 (1995), the entirety of which is herein incorporated by reference; Shalon, Ph.D. Thesis. Stanford University (1996), the entirety of which is herein incorporated by reference). Every nucleotide in a large sequence can be queried at the same time. Hybridization can be used to efficiently analyze nucleotide sequences. Such microarrays can be probed with any combination of nucleic acid molecules. Particularly preferred combinations of nucleic acid molecules to be used as probes include a population of mRNA molecules from a known tissue type or a known developmental stage or a plant subject to a known stress (environmental or man-made) or any combination thereof (e.g. mRNA made from water stressed leaves at the 2 leaf stage). Expression profiles generated by this method can be utilized as markers.

The genetic linkage of additional marker molecules can be established by a gene mapping model such as, without limitation, the flanking marker model reported by Lander and Botstein, Genetics, 121:185-199 (1989), and the interval mapping, based on maximum likelihood methods described by Lander and Botstein, Genetics, 121:185-199 (1989), and implemented in the software package MAPMAKER/QTL (Lincoln and Lander, Mapping Genes Controlling Quantitative Traits Using MAPMAKER/QTL, Whitehead Institute for Biomedical Research, Mass., (1990). Additional software includes Qgene, Version 2.23 (1996), Department of Plant Breeding and Biometry, 266 Emerson Hall, Cornell University, Ithaca, N.Y., the manual of which is herein incorporated by reference in its entirety). Use of Qgene software is a particularly preferred approach.

A maximum likelihood estimate (MLE) for the presence of a marker is calculated, together with an MLE assuming no QTL effect, to avoid false positives. A logio of an odds ratio (LOD) is then calculated as: LOD=log₁₀ (MLE for the presence of a QTL/MLE given no linked QTL).

The LOD score essentially indicates how much more likely the data are to have arisen assuming the presence of a QTL than in its absence. The LOD threshold value for avoiding a false positive with a given confidence, say 95%, depends on the number of markers and the length of the genome. Graphs indicating LOD thresholds are set forth in Lander and Botstein, Genetics, 121:185-199 (1989), and further described by Arus and Moreno-González, Plant Breeding, Hayward, Bosemark, Romagosa (eds.) Chapman & Hall, London, pp. 314-331 (1993).

Additional models can be used. Many modifications and alternative approaches to interval mapping have been reported, including the use non-parametric methods (Kruglyak and Lander, Genetics, 139:1421-1428 (1995), the entirety of which is herein incorporated by reference). Multiple regression methods or models can be also be used, in which the trait is regressed on a large number of markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given marker interval, and at the same time onto a number of markers that serve as ‘cofactors,’ have been reported by Jansen and Stam, Genetics, 136:1447-1455 (1994) and Zeng, Genetics, 136:1457-1468 (1994). Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp.195-204 (1994), thereby improving the precision and efficiency of QTL mapping (Zeng, Genetics, 136:1457-1468 (1994)). These models can be extended to multi-environment experiments to analyze genotype-environment interactions (Jansen et al., Theo. Appl. Genet. 91:33-37 (1995).

Selection of an appropriate mapping populations is important to map construction. The choice of appropriate mapping population depends on the type of marker systems employed (Tanksley et al., Molecular mapping plant chromosomes. chromosome structure and function: Impact of new concepts J. P. Gustafson and R. Appels (eds.). Plenum Press, New York, pp. 157-173 (1988), the entirety of which is herein incorporated by reference). Consideration must be given to the source of parents (adapted vs. exotic) used in the mapping population. Chromosome pairing and recombination rates can be severely disturbed (suppressed) in wide crosses (adapted x exotic) and generally yield greatly reduced linkage distances. Wide crosses will usually provide segregating populations with a relatively large array of polymorphisms when compared to progeny in a narrow cross (adapted×adapted).

An F₂ population is the first generation of selfing after the hybrid seed is produced. Usually a single F₁ plant is selfed to generate a population segregating for all the genes in Mendelian (1:2:1) fashion. Maximum genetic information is obtained from a completely classified F₂ population using a codominant marker system (Mather, Measurement of Linkage in Heredity: Methuen and Co., (1938), the entirety of which is herein incorporated by reference). In the case of dominant markers, progeny tests (e.g F₃, BCF₂) are required to identify the heterozygotes, thus making it equivalent to a completely classified F₂ population. However, this procedure is often prohibitive because of the cost and time involved in progeny testing. Progeny testing of F₂ individuals is often used in map construction where phenotypes do not consistently reflect genotype (e.g. disease resistance) or where trait expression is controlled by a QTL. Segregation data from progeny test populations (e.g. F₃ or BCF₂) can be used in map construction. Marker-assisted selection can then be applied to cross progeny based on marker-trait map associations (F₂, F₃), where linkage groups have not been completely disassociated by recombination events (i.e., maximum disequilibrium).

Recombinant inbred lines (RIL) (genetically related lines; usually >F₅, developed from continuously selfing F₂ lines towards homozygosity) can be used as a mapping population. Information obtained from dominant markers can be maximized by using RIL because all loci are homozygous or nearly so. Under conditions of tight linkage (i.e., about <10% recombination), dominant and co-dominant markers evaluated in RIL populations provide more information per individual than either marker type in backcross populations (Reiter et al., Proc. Natl. Acad. Sci. (U.S.A.) 89:1477-1481 (1992)). However, as the distance between markers becomes larger (i.e., loci become more independent), the information in RIL populations decreases dramatically when compared to codominant markers.

Backcross populations (e.g., generated from a cross between a successful variety (recurrent parent) and another variety (donor parent) carrying a trait not present in the former) can be utilized as a mapping population. A series of backcrosses to the recurrent parent can be made to recover most of its desirable traits. Thus a population is created consisting of individuals nearly like the recurrent parent but each individual carries varying amounts or mosaic of genomic regions from the donor parent. Backcross populations can be useful for mapping dominant markers if all loci in the recurrent parent are homozygous and the donor and recurrent parent have contrasting polymorphic marker alleles (Reiter et al., Proc. Natl. Acad. Sci. (U.S.A.) 89:1477-1481 (1992)). Information obtained from backcross populations using either codominant or dominant markers is less than that obtained from F₂ populations because one, rather than two, recombinant gametes are sampled per plant. Backcross populations, however, are more informative (at low marker saturation) when compared to RILs as the distance between linked loci increases in RIL populations (i.e. about 0.15% recombination). Increased recombination can be beneficial for resolution of tight linkages, but may be undesirable in the construction of maps with low marker saturation.

Near-isogenic lines (NIL) created by many backcrosses to produce an array of individuals that are nearly identical in genetic composition except for the trait or genomic region under interrogation can be used as a mapping population. In mapping with NILs, only a portion of the polymorphic loci are expected to map to a selected region.

Bulk segregant analysis (BSA) is a method developed for the rapid identification of linkage between markers and traits of interest (Michelmore, et al., Proc. Natl. Acad. Sci. (U.S.A.) 88:9828-9832 (1991)). In BSA, two bulked DNA samples are drawn from a segregating population originating from a single cross. These bulks contain individuals that are identical for a particular trait (resistant or susceptible to particular disease) or genomic region but arbitrary at unlinked regions (i.e. heterozygous). Regions unlinked to the target region will not differ between the bulked samples of many individuals in BSA.

The markers of the present invention can be used to isolate or substantially purify an allele of a quantitative trait locus that is also located on linkage group U03 of a Glycine maxPI290136 plant. Construction of an overlapping series of clones (a clone contig) across the region can provide the basis for a physical map encompassing an allele of a quantitative trait locus that are located on linkage group U03 of a Glycine max PI290136 plant. The yeast artificial chromosome (YAC) cloning system has facilitated chromosome walking and large-size cloning strategies. A sequence tag site (STS) content approach utilizing the markers of the present invention can be used for the construction of YAC clones across chromosome regions. Such an STS content approach to the construction of YAC maps can provide a detailed and ordered STS-based map of any chromosome region, including the region encompassing the allele of a quantitative trait locus is also located on linkage group U03 of a Glycine max PI290136 plant. YAC maps can be supplemented by detailed physical maps are constructed across the region by using BAC, PAC, or bacteriophage P1 clones that contain inserts ranging in size from 70 kb to several hundred kilobases (Cregan, Theor. Appl.Gen. 78:919-928 (1999), Sternberg, Proc. Natl. Acad. Sci. 87:103-107 (1990), Stemberg, Trends Genet. 8:11-16 (1992); Sternberg et al., New Biol. 2:151-162 (1990); Ioannou et al., Nat. Genet. 6:84-89 (1994); Shizuya et al., Proc. Natl. Acad. Sci. 89:8794-8797 (1992), all of which are herein incorporated by reference in their entirety).

Overlapping sets of clones are derived by using the available markers of the present invention to screen BAC, PAC, bacteriophage PI, or cosmid libraries. In addition, hybridization approaches can be used to convert the YAC maps into BAC, PAC, bacteriophage P 1, or cosmid contig maps. Entire YACs and products of inter-Alu-PCR as well as primer sequences from appropriate STSs can be used to screen BAC, PAC, bacteriophage P1, or cosmid libraries. The clones isolated for any region can be assembled into contigs using STS content information and fingerprinting approaches (Sulston et al., Comput. Appl. Biosci. 4:125-132 (1988)).

The degeneracy of the genetic code, which allows different nucleic acid sequences to code for the same protein or peptide, is known in the literature. As used herein a nucleic acid molecule is degenerate of another nucleic acid molecule when the nucleic acid molecules encode for the same amino acid sequences but comprise different nucleotide sequences. An aspect of the present invention is that the nucleic acid molecules of the present invention include nucleic acid molecules that are degenerate of the nucleic acid molecule that encodes the protein(s) of the quantitative trait alleles.

Another aspect of the present invention is that the nucleic acid molecules of the present invention include nucleic acid molecules that are homologues of the nucleic acid molecule that encodes the one or more of the proteins associated with the quantitative trait locus.

Exogenous genetic material may be transferred into a plant by the use of a DNA plant transformation vector or construct designed for such a purpose. A particularly preferred subgroup of exogenous material comprises a nucleic acid molecule of the present invention. Design of such a vector is generally within the skill of the art (See, Plant Molecular Biology: A Laboratory Manual, eds. Clark, Springer, New York (1997), the entirety of which is herein incorporated by reference). Examples of such plants, include, without limitation, alfalfa, Arabidopsis, barley, Brassica, broccoli, cabbage, citrus, cotton, garlic, oat, oilseed rape, onion, canola, flax, maize, an ornamental plant, pea, peanut, pepper, potato, rice, rye, sorghum, soybean, strawberry, sugarcane, sugarbeet, tomato, wheat, poplar, pine, fir, eucalyptus, apple, lettuce, lentils, grape, banana, tea, turf grasses, sunflower, oil palm, Phaseolus etc.

A construct or vector may include the endogenous promoter of the enhanced yield QTL of the present invention or a heterologous promoter may be selected to express the protein or protein fragment of choice. A number of promoters which are active in plant cells have been described in the literature. These include the nopaline synthase (NOS) promoter (Ebert et al., Proc. Natl. Acad. Sci. (U.S.A.) 84:5745-5749 (1987), the entirety of which is herein incorporated by reference), the octopine synthase (OCS) promoter (which are carried on tumor-inducing plasmids of Agrobacterium tumefaciens), the caulimovirus promoters such as the cauliflower mosaic virus (CaMV) 19S promoter (Lawton et al., Plant Mol. Biol. 9:315-324 (1987), the entirety of which is herein incorporated by reference) and the CaMV 35S promoter (Odell et al., Nature 313:810-812 (1985), the entirety of which is herein incorporated by reference), the figwort mosaic virus 35S-promoter, the light-inducible promoter from the small subunit of ribulose-1,5-bis-phosphate carboxylase (ssRUBISCO), the Adh promoter (Walker et al., Proc. Natl. Acad. Sci. (U.S.A.) 84:6624-6628 (1987), the entirety of which is herein incorporated by reference), the sucrose synthase promoter (Yang et al., Proc. Natl. Acad. Sci. (U.S.A.) 87:4144-4148 (1990), the entirety of which is herein incorporated by reference), the R gene complex promoter (Chandler et al., The Plant Cell 1:1175-1183 (1989), the entirety of which is herein incorporated by reference), and the chlorophyll a/b binding protein gene promoter, etc. These promoters have been used to create DNA constructs which have been expressed in plants; see, e.g., PCT publication WO 84/02913, herein incorporated by reference in its entirety.

Promoters which are known or are found to cause transcription of DNA in plant cells can be used in the present invention. Such promoters may be obtained from a variety of sources such as plants and plant viruses. In addition to promoters that are known to cause transcription of DNA in plant cells, other promoters may be identified for use in the current invention by screening a plant cDNA library for genes which are selectively or preferably expressed in the target tissues or cells.

Constructs or vectors may also include with the coding region of interest a nucleic acid sequence that acts, in whole or in part, to terminate transcription of that region. For example, such sequences have been isolated including the Tr73′ sequence and the NOS 3′ sequence (Ingelbrecht et al., The Plant Cell 1:671-680 (1989), the entirety of which is herein incorporated by reference; Bevan et al., Nucleic Acids Res. 11:369-385 (1983), the entirety of which is herein incorporated by reference), or the like.

A vector or construct may also include regulatory elements. Examples of such include the Adh intron 1 (Callis et al., Genes and Develop. 1:1183-1200 (1987), the entirety of which is herein incorporated by reference), the sucrose synthase intron (Vasil et al., Plant Physiol. 91:1575-1579 (1989), the entirety of which is herein incorporated by reference) and the TMV omega element (Gallie et al., The Plant Cell 1:301-311 (1989), the entirety of which is herein incorporated by reference). These and other regulatory elements may be included when appropriate.

A vector or construct may also include a selectable marker. Selectable markers may also be used to select for plants or plant cells that contain the exogenous genetic material. Examples of such include, but are not limited to, a neo gene (Potrykus et al., Mol. Gen. Genet. 199:183-188 (1985), the entirety of which is herein incorporated by reference) which codes for kanamycin resistance and can be selected for using kanamycin, G418, etc.; a bar gene which codes for bialaphos resistance; a mutant EPSP synthase gene (Hinchee et al., Bio/Technology 6:915-922 (1988), the entirety of which is herein incorporated by reference) which encodes glyphosate resistance; a nitrilase gene which confers resistance to bromoxynil (Stalker et al., J. Biol. Chem. 263:6310-6314 (1988), the entirety of which is herein incorporated by reference); a mutant acetolactate synthase gene (ALS) which confers imidazolinone or sulphonylurea resistance (European Patent Application 154,204 (Sep. 11, 1985), the entirety of which is herein incorporated by reference); and a methotrexate resistant DHFR gene (Thillet et al., J. Biol. Chem. 263:12500-12508 (1988), the entirety of which is herein incorporated by reference).

A vector or construct may also include a screenable marker. Screenable markers may be used to monitor expression. Exemplary screenable markers include a β-glucuronidase or uidA gene (GUS) which encodes an enzyme for which various chromogenic substrates are known (Jefferson, Plant Mol. Biol, Rep. 5:387-405 (1987), the entirety of which is herein incorporated by reference; Jefferson et al., EMBO J. 6:3901-3907 (1987), the entirety of which is herein incorporated by reference); an R-locus gene, which encodes a product that regulates the production of anthocyanin pigments (red color) in plant tissues (Dellaporta et al., Stadler Symposium 11:263-282 (1988), the entirety of which is herein incorporated by reference); a β-lactamase gene (Sutcliffe et al., Proc. Natl. Acad. Sci. (U.S.A.) 75:3737-3741 (1978), the entirety of which is herein incorporated by reference), a gene which encodes an enzyme for which various chromogenic substrates are known (e.g., PADAC, a chromogenic cephalosporin); a luciferase gene (Ow et al., Science 234:856-859 (1986), the entirety of which is herein incorporated by reference); a xylE gene (Zukowsky et al., Proc. Natl. Acad. Sci. (U.S.A.) 80:1101-1105 (1983), the entirety of which is herein incorporated by reference) which encodes a catechol dioxygenase that can convert chromogenic catechols; an β-amylase gene (Ikatu et al., Bio/Technol. 8:241-242 (1990), the entirety of which is herein incorporated by reference); a tyrosinase gene (Katz et al., J. Gen. Microbiol. 129:2703-2714 (1983), the entirety of which is herein incorporated by reference) which encodes an enzyme capable of oxidizing tyrosine to DOPA and dopaquinone which in turn condenses to melanin; and an β-galactosidase.

There are many methods for introducing transforming nucleic acid molecules into plant cells. Suitable methods are believed to include virtually any method by which nucleic acid molecules may be introduced into a cell, such as by Agrobacterium infection or direct delivery of nucleic acid molecules such as, for example, by PEG-mediated transformation, by electroporation or by acceleration of DNA coated particles, etc. (Potrykus, Ann. Rev. Plant Physiol. Plant Mol. Biol. 42:205-225 (1991), the entirety of which is herein incorporated by reference; Vasil, Plant Mol. Biol. 25:925-937 (1994), the entirety of which is herein incorporated by reference). For example, electroporation has been used to transform Zea mays protoplasts (Fromm et al., Nature 312:791-793 (1986), the entirety of which is herein incorporated by reference).

Other vector systems suitable for introducing transforming DNA into a host plant cell include but are not limited to binary artificial chromosome (BIBAC) vectors (Hamilton et al., Gene 200:107-116 (1997), the entirety of which is herein incorporated by reference), and transfection with RNA viral vectors (Della-Cioppa et al., Ann. N. Y. Acad. Sci. (1996), 792 (Engineering Plants for Commercial Products and Applications), 57-61, the entirety of which is herein incorporated by reference.

Technology for introduction of DNA into cells is well known to those of skill in the art. Four general methods for delivering a gene into cells have been described: (1) chemical methods (Graham and van der Eb, Virology 54:536-539 (1973), the entirety of which is herein incorporated by reference); (2) physical methods such as microinjection (Capecchi, Cell 22:479-488 (1980), the entirety of which is herein incorporated by reference), electroporation (Wong and Neumann, Biochem. Biophys. Res. Commun. 107:584-587 (1982); Fromm et al., Proc. Natl. Acad. Sci. (U.S.A.) 82:5824-5828 (1985); U.S. Pat. No. 5,384,253, all of which are herein incorporated in their entirety); and the gene gun (Johnston and Tang, Methods Cell Biol. 43:353-365 (1994), the entirety of which is herein incorporated by reference); (3) viral vectors (Clapp, Clin. PerinatoL 20:155-168 (1993); Lu et al., J. Exp. Med. 178:2089-2096 (1993); Eglitis and Anderson, Biotechniques 6:608-614 (1988), all of which are herein incorporated in their entirety); and (4) receptor-mediated mechanisms (Curiel et al., Hum. Gen. Ther. 3:147-154 (1992), Wagner et al., Proc. Natl. Acad. Sci. USA 89:6099-6103 (1992), all of which are incorporated by reference in their entirety).

Acceleration methods that may be used include, for example, microprojectile bombardment and the like. One example of a method for delivering transforming nucleic acid molecules to plant cells is microprojectile bombardment. This method has been reviewed by Yang and Christou, eds., Particle Bombardment Technology for Gene Transfer, Oxford Press, Oxford, England (1994), the entirety of which is herein incorporated by reference. Non-biological particles (microprojectiles) that may be coated with nucleic acids and delivered into cells by a propelling force. Exemplary particles include those comprised of tungsten, gold, platinum, and the like.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example the methods described by Fraley et al., Bio/Technology 3:629-635 (1985) and Rogers et al., Methods Enzymol. 153:253-277 (1987), both of which are herein incorporated by reference in their entirety. Further, the integration of the Ti-DNA is a relatively precise process resulting in few rearrangements. The region of DNA to be transferred is defined by the border sequences, and intervening DNA is usually inserted into the plant genome as described (Spielmann et al., Mol. Gen. Genet. 205:34 (1986), the entirety of which is herein incorporated by reference).

A transgenic plant formed using Agrobacterium transformation methods typically contains a single gene on one chromosome. Such transgenic plants can be referred to as being hemizygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants produced for the gene of interest.

It is also to be understood that two different transgenic plants can also be mated to produce offspring that contain two independently segregating added, exogenous genes. Selfing of appropriate progeny can produce plants that are homozygous for both added, exogenous genes that encode a polypeptide of interest. Back-crossing to a parental plant and out-crossing with a non-transgenic plant are also contemplated, as is vegetative propagation.

The regeneration, development, and cultivation of plants from single plant protoplast transformants or from various transformed explants is well known in the art (Weissbach and Weissbach, In: Methods for Plant Molecular Biology, (Eds.), Academic Press, Inc. San Diego, Calif., (1988), the entirety of which is herein incorporated by reference). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a protein of interest is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

Methods for transforming dicots, primarily by use of Agrobacterium tumefaciens, and obtaining transgenic plants have been published for cotton (U.S. Pat. Nos. 5,004,863, 5,159,135, 5,518,908, all of which are herein incorporated by reference in their entirety); soybean (U.S. Pat. Nos. 5,569,834, 5,416,011, McCabe et. al., Bio/Technology 6:923 (1988), Christou et al., Plant Physiol. 87:671-674 (1988), all of which are herein incorporated by reference in their entirety); Brassica (U.S. Pat. No. 5,463,174, the entirety of which is herein incorporated by reference); peanut (Cheng et al., Plant Cell Rep. 15:653-657 (1996), McKently et al., Plant Cell Rep. 14:699-703 (1995), all of which are herein incorporated by reference in their entirety); papaya; and pea (Grant et al., Plant Cell Rep. 15:254-258, (1995), the entirety of which is herein incorporated by reference).

Transformation of monocotyledons using electroporation, particle bombardment, and Agrobacterium have also been reported. Transformation and plant regeneration have been achieved in asparagus (Bytebier et al., Proc. Natl. Acad. Sci. (USA) 84:5354, (1987), the entirety of which is herein incorporated by reference); barley (Wan and Lemaux, Plant Physiol 104:37 (1994), the entirety of which is herein incorporated by reference); Zea mays (Rhodes et al., Science 240:204 (1988), Gordon-Kamm et al., Plant Cell 2:603-618 (1990), Fromm et al., Bio/Technology 8:833 (1990), Koziel et al., gBio/Technology 11:194, (1993), Armstrong et al., Crop Science 35:550-557 (1995), all of which are herein incorporated by reference in their entirety); oat (Somers et al., Bio/Technology 10:1589 (1992), the entirety of which is herein incorporated by reference); orchard grass (Horn et al., Plant Cell Rep. 7:469 (1988), the entirety of which is herein incorporated by reference); rice (Toriyama et al., Theor Appl. Genet. 205:34, (1986); Part et al., Plant Mol. Biol. 32:1135-1148, (1996); Abedinia et al., Aust. J. Plant Physiol. 24:133-141 (1997); Zhang and Wu, Theor. Appl. Genet. 76:835 (1988); Zhang et al. Plant Cell Rep. 7:379, (1988); Battraw and Hall, Plant Sci. 86:191-202 (1992); Christou et al., Bio/Technology 9:957 (1991), all of which are herein incorporated by reference in their entirety); rye (De la Pena et al., Nature 325:274 (1987), the entirety of which is herein incorporated by reference); sugarcane (Bower and Birch, Plant J. 2:409 (1992), the entirety of which is herein incorporated by reference); tall fescue (Wang et al., Bio/Technology 10:691 (1992), the entirety of which is herein incorporated by reference), and wheat (Vasil et al., Bio/Technology 10:667 (1992), the entirety of which is herein incorporated by reference; U.S. Pat. No. 5,631,152, the entirety of which is herein incorporated by reference.).

In addition to the above discussed procedures, practitioners are familiar with the standard resource materials which describe specific conditions and procedures for the construction, manipulation and isolation of macromolecules (e.g., DNA molecules, plasmids, etc.), generation of recombinant organisms and the screening and isolating of clones, (see for example, Sambrook et al., Molecular Cloning: A Laboratory Manual, Cold Spring Harbor Press (1989); Mailga et al., Methods in Plant Molecular Biology, Cold Spring Harbor Press (1995), the entirety of which is herein incorporated by reference; Birren et al., Genome Analysis: Detecting Genes, 1, Cold Spring Harbor, N.Y. (1998), the entirety of which is herein incorporated by reference; Birren et al., Genome Analysis: Analyzing DNA, 2, Cold Spring Harbor, N.Y. (1998), the entirety of which is herein incorporated by reference; Plant Molecular Biology: A Laboratory Manual, eds. Clark, Springer, New York (1997), the entirety of which is herein incorporated by reference).

Having now generally described the invention, the same will be more readily understood through reference to the following examples which are provided by way of illustration, and are not intended to be limiting of the present invention, unless specified.

### EXAMPLE 1

Two leaf discs are collected (approximately 40 mg) from a healthy leaf of a young Glycine max plant and stored on wet ice or at 4° C. Tissue samples are then freeze-dried and stored at −20° C. or −80° C. The frozen samples are kept as dry as possible and sealed from contact with the atmosphere. The freeze-dried samples from −20° C. or −80° C., are allowed to warm up to room temperature prior to unsealing or opening. One leaflet (or 2 leaf discs) is inserted into an 1.5 ml Eppendorf tube, placed on dry ice, and crushed with a wooden dowel. Approximately 200 μl of microprep buffer (25 ml extraction buffer (350 mM sorbitol, 100 mM Tris-base, 5 mM EDTA-Na₂), 25 ml nuclei lysis buffer (IM Tris/HCl, 0.5 M EDTA, 5 M NaCl, 2% CTAB), 10 ml 5% sarkosyl, 0.1 g Na bisulfite) is added to each sample. The sample is then homogenized. An additional 550 μl of microprep buffer is added, mixed by vortex for about 30-60 seconds, and incubated at 65° C. for about 60 minutes. About 700 μl chloroform/isoamyl alcohol (24:1) is added, mixed well for about 10-30 seconds. Centrifugation of the tubes is performed at approximately 10,000 rpm for 5 minutes in a microcentrifuge. The aqueous phase is transferred into a new tube and RNA is removed from the extract by the addition of 30 μl of RNase (10 mg/ml) to the aqueous phase and incubated for 1 hour at room temperature. Approximately 500 μl ice-cold isopropanol is added to the aqueous extract, and the tubes inverted until the DNA precipitated. The precipitated solution is kept at 4° C. for about 1 hour or overnight. Centrifugation of the tubes is performed at approximately 10,000 rpm for 5 minutes in a microcentrifuge. The supernatant is discarded and the pellet washed 1-3 times with 200 μl 70% ethanol. The ethanol is removed using a micropipette and pellet dried at 37° C. for 10 minutes. The DNA is dissolved in 50 μl TE (10 mM Tris-HCL pH8.0, 0.1 mM EDTA), then kept overnight at 4° C. Centrifugation of the tubes is performed at approximately 10,000 rpm for 5 minutes and then the supernatant is transferred into new tubes. Using this method approximately 2 μg of DNA per mg of fresh leaf tissue is extracted.

The amount of DNA recovered is quantified by performing agarose gel electrophoresis on aliquots of the DNA extracted from the samples. The agarose gel is prepared as follows: 4 g agarose melted 400 ml 1× TBE (89 mM Tris-HCl, 89 mM boric acid, 2 mM EDTA), cooled to ˜70° C. and then 10 μl of 10 mg/ml ethidium bromide is added to the gel. A gel mold with comb for sample application is prepared and molten agarose poured into the mold. After the gel has solidified it is transferred to the electrophoresis apparatus containing approximately 2 L of 1× TBE buffer. For each sample, 9 μl (1 μl sample, 1μl loading buffer with marker dye (50% glycerol, 0.1M EDTA, 0.1% bromophenol blue), 7 μl TE) is loaded. Molecular weight standards are included in the gel. The electrophoresis is conducted at approximately 100 mA for 2-4 hrs. The DNA concentration in each sample is estimated by it's staining intensity relative to the standards. The volume of the DNA sample is adjusted with 1 X TE such that the concentration of the DNA in each sample is about 1 ng/μl.

For each sample a 5 μl aliquot is placed into each well of a Perkin-Elmer MicroAmp Optical 96 Well reaction plates, to which is added 1.5 μl H20, 1.0 μl 10× PCR buffer, 0.04 μl 25 mM dNTPs, 1.0 Dye Dye (20 mM MgCl2, 20% sucrose, 1 mM Cresol Red), 1.5 μl 1M mix of forward and reverse primers for each SSR marker, and 0.064 μl of 0.32 units of Taq polymerase. The marker pairs are SEQ ID NO. 1 and SEQ ID NO. 2 for SATT315; SEQ ID NO. 3 and SEQ ID NO 4 for SATT187; SEQ ID NO 5 and SEQ ID NO 6 for SCNB188; SEQ ID NO 7 and SEQ ID NO 8 for Sy50; SEQ ID NO 9 and SEQ ID NO 10 for SCNB187; SEQ ID NO 11 and SEQ ID NO 12 for Sy36; SEQ ID NO 13 and SEQ ID NO 14 for SCNB190; SEQ ID NO 15 and SEQ ID NO 16 for SAT 212; SEQ ID NO 17 and SEQ ID NO 18 for SAT_{—}215, Table 1. Polymerase chain reaction is performed with the following thermal cycler conditions, 94° C. 4 minutes.; 94° C. 25 sec., 47° C. 25 sec., 72° C. 25 sec., 32 cycles; 72° C. 3 minutes for final extension and 4° C. hold.

An acrylamide gel is prepared using 56.5 ml water, 3.5 ml 10× TAE buffer, 10.5 ml 40% acrylamide stock solution, 50 μl TEMED, 0.06 g ammonium persulfate. A total of 5 μl of the PCR product is loaded onto the acrylamide gels on 1× TAE buffer. Molecular weight ladders are also loaded onto the gel to facilitate identification of SSR bands. Gels are run at for 45 minutes at 300V. The electrophoresis is stopped when the cresol red dye is at the bottom of the gel. Gels are then stained with SYBR green by mixing 20 μl of 10,000× SYBR green and 200 ml 1× TAE buffer. The mixture should be enough to stain 20 gels. Gels are stained for 15-20 minutes with vigorous shaking. The gel bands are then visualized under a UV transilluminator. The PCR reaction product is then scored for the presence or absence of the bands on the appropriate molecular weights of SSR markers spanning the Sy5 yield QTL. The DNA sequence analysis of the PCR products are shown in SEQ ID NOs: 19-25.

TABLE 1
SSR primer sequences for molecular markers of the Sy5 locus

| SSR | | |
|---|---|---|
| LOCUS | FORWARD PRIMER | REVERSE PRIMER |
| Satt315 | GCGCGACAACTCTAATGAAAATCT | GCGGAGTTTGATTTTTCAAAAGT |
| Satt187 | GCGTTTTAATTTATGATATAACCAA | GCGTTTTATCTCTTTTTCCACAAC |
| SCNB188 | ATCAATCGACGCAATAATCAAGAAA | ATGATGAGAAGACAATGGGATGTCA |
| Sy50 | CAGGCTTCAGTGTGCATAATACAGG | TTCTATGTTCCCTGTGCAAACACTG |
| SCNB187 | GTCTGCAAGCTAACAGTGTCAGAGG | CACACTCAATCTCATTAGCAGACACG |
| Sy36 | TCCTTTGGCTCACTATTGACGATTT | ACCCGTGTGCCACTTTAACTACATT |
| SCNB190 | TAACGCTGCATGATTTGAGTTCTGT | GTATTGGTTGGACTTTGGAGACCAC |
| Sat_212 | GCGGACAATTTTTTATCAATAATTTATT | GCGATGCTTACTTTTCCTATGATCACTT |
| Sat_215 | GCGTAGCAACAAAGCAATCTACAG | GCGTCCCATTTTATTCCACACTATGTAAT |

### EXAMPLE 2

Glycine max PI290136 or a black seeded donor parent carrying the Sy5 yield locus is crossed between parent soybean line H5050 (Hartz Seed, Stuttgart, Ark.) and soybean line CX445 (DeKalb® Seed, DeKalb Ill.) and is selected for the Sy5 yield locus and yellow seed color by the protocol shown in Table 2a and 2b.

TABLE 2a
Isoline development for breaking linkage between Sy5 locus and black
seed color

| | | |
|---|---|---|
| A | D | Cross elite, yellow seed coat Asgrow lines (A) to |
| | | black seeded donor parent carrying Sy5 QTL. |
| F₁ | A | Cross F₁, which is heterozygous throughout |
| | | the Sy5 region to the |
| | | black seeded donor parent |
| BC₁F₁ | A | BC₁F₁ plants segregate at a 1:1 ratio for |
| | | elite (Asgrow line) |
| | | and donor parent alleles. Genotype BC₁F₁ |
| | | plants with 2 SSR |
| | | markers flanking Sy5 (positions based on |
| | | QTL mapping results). |
| | | The region between SSR markers covers |
| | | approximately 15 cM. |
| | | Select individuals that are heterozygous |
| | | for both flanking markers and cross |
| | | to the black seeded donor. |
| BC₂F₁ | | BC₂F₁ plants segregate 1:1 in the Sy5 |
| | | region because all BC₁F₁ |
| | | parents are heterozygous. Genotype BC₂F₁ |
| | | plants with the same |
| | | 2 SSR markers flanking Sy5 that are used in the |
| | | BC₁F₁ and |
| | | identify individuals that are heterozygous |
| | | for both flanking markers. |
| BC₂F₂ | | All BC₂F₁ parents are |
| | | heterozygous throughout the Sy5 region. |
| | | Genotype BC₂F₂ plants with four |
| | | SSR markers in the Sy5 |
| | | region and identify plants heterozygous at |
| | | all four marker |
| | | loci. Any individuals that could not be confirmed |
| | | as heterozygotes at all four loci are discarded. |
| | | Self pollinated seed is harvested |
| | | in bulk from the heterozygotes. |
| BC₂F₃ | | Seed available for planting BC₂F₃ plants, all of |
| | | which are obtained by selfing individuals |
| | | heterozygous throughout the Sy5 |
| | | region (heterozygous at 4 SSR loci). |
| | | The BC₂F₃ generation will |
| | | segregate in a 1:2:1 ratio at the I locus (II:Ii:ii). |
| | | Seed will be |
| | | harvested from yellow plants, (II and Ii), |
| | | which will segregate in a 1:2 ratio (II:Ii). |
| BC₂F_{3:4} | | Plant F_{3:4} lines derived from |
| | | yellow seed coat parents. Identify |
| | | nonsegregating rows for seed color, |
| | | which arose from homozygous yellow parents. |
| | | Yellow seed coat parents segregated 2:1 (Ii:II), |
| | | hence, 1/3 of BC₂F_{3:4} rows |
| | | will be uniformly yellow seed. |
| | | BC₂F₃ plants genotyped using |
| | | flanking SSR markers. Desired |
| | | BC₂F₃ plants carry one parental |
| | | gamete throughout the Sy5 region |
| | | and one recombinant gamete. |

TABLE 2b
The gametic array resulting from a yellow seeded homozygote

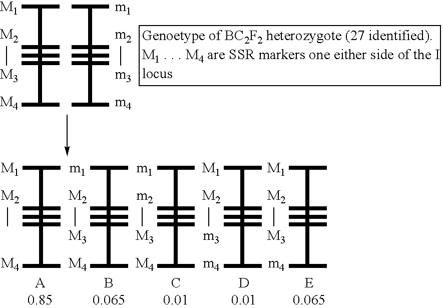

Array of gametes carrying a dominant ‘I’ allele in the BC₂F₃ generation produced by a heterozygous BC₂F₂ parent. Assume a recombination frequency of 0.065 between M₁ and I and between M₄, and I. Assume a recombination frequency of 0.01 between M₂ and I and between M₃ and I. The frequencies of each type of gamete are given below the gametes.
# Total recombination frequency between M₁ and M₄ is 0.15. Gamete ‘A’ is a parental gamete, while B..E are recombinant gametes. Gametes C and D had recombinants between I and one of the closely flanking markers.

BC₂F₃:₄ lines are desired that arose from BC₂F₃ individuals that are homozygous yellow seed coat and contain one parental gamete and one recombinant gamete close to the I locus (gametes C and D above). Homozygous yellow BC₂F₃ individuals are the result of randomly sampling two gametes of types A.E. Explicit frequencies of all possible BC₂F₃ genotypes can be found in a 5×5 Punnett square given in Table 2c. Desired individuals carry a single parental gamete (A) and a gamete with a recombination between M₂ and I or between M₃ and I. Four cells in Table 2c correspond to the desired type, each with a frequency of 0.0085. The total frequency of individuals with one parental and one recombinant gamete is 4×0.0085=0.034.

Starting with the plants available in the BC₂F₃ generation, 0.25 will be homozygous yellow, of which 0.034 will contain one parental and one recombinant gamete. The total frequency of desired BC₂F₃ plants will be 0.0085. Assuming 2000 homozygous yellow rows in the BC₂F_{3:4} generation, the number of desired rows would be 2000×0.33×0.034=22.7 individuals. Carrying the frequency of desired BC₂F_{3:4} rows one step further, r=number recombination fraction between M₁ and M₄, r₁=recombination fraction between M₂ and I, and M₃=recombination fraction between M₃ and I. Then, out of 2000 rows, the number with the desired genotype will be: desired=2000×⅓×2(1r)(r₁+r₂).

TABLE 2c
Punnett square giving frequencies of BC₂F₃ genotypes carrying two copies
of the dominant yellow allele

| | A | B | C | D | E |
|---|---|---|---|---|---|
| A(0.85) | 0.7225 | 0.05525 | 0.0085 | 0.0085 | 0.05525 |
| B(0.065) | 0.05525 | 0.004225 | 0.00065 | 0.00065 | 0.004225 |
| C(0.01) | 0.0085 | 0.00065 | 0.0001 | 0.0001 | 0.00065 |
| D(0.01) | 0.0085 | 0.00065 | 0.0001 | 0.0001 | 0.00065 |
| E(0.065) | 0.05525 | 0.004225 | 0.00065 | 0.00065 | 0.004225 |

Pollen from the F₁ progeny of that cross are then crossed back to the parent line to generate about 40 BC₁F₁ progeny. Each BC₁F₁ progeny is then grown and crossed again to the parent line to generate between 250 and 300 BC₂F₁ progeny. The BC₂F₁ progeny are grown and leaf samples are taken from each plant for subsequent DNA extraction and molecular marker genotyping. The BC₂F₁ plants are grown to maturity and genotyped with the molecular markers flanking the Sy5 locus. Nine BC₂F₁ heterozygote lines for both flanking markers are identified (Table 3). The BC₂F₂ seeds are collected from each BC₂F₁ plant then bulked. The resulting seeds from each of BC₂F₁-derived progeny are used for yield field trials.

TABLE 3
Backcrossed populations containing yellow seeded Glycine max with the
Sy5 yield locus

| 9 BC2F1Heterozygotes | 27 BC2F2Heterozygotes |
|---|---|
| Sy5BC2F1AG3002-29 | Sy5BC2F2AG3002-164 |
| Sy5BC2F1AG3002-34 | Sy5BC2F2AG3002-186 |
| Sy5BC2F1AG3002-35 | Sy5BC2F2AG3002-200 |
| Sy5BC2F1AG3002-36 | Sy5BC2F2AG3002-209 |
| Sy5BC2F1AG3002-40 | Sy5BC2F2AG3002-354 |
| Sy5BC2F1AG3002-41 | Sy5BC2F2AG3002-376 |
| Sy5BC2F1AG3002-43 | Sy5BC2F2AG3002-415 |
| Sy5BC2F1AG23701-66 | Sy5BC2F2AG3002-457 |
| Sy5BC2F1AG23701-69 | Sy5BC2F2AG3002-481 |
| | Sy5BC2F2AG3002-514 |
| | Sy5BC2F2AG3002-598 |
| | Sy5BC2F2AG3002-607 |
| | Sy5BC2F2AG3002-720 |
| | Sy5BC2F2AG3002-737 |
| | Sy5BC2F2AG3002-770 |
| | Sy5BC2F2AG3002-795 |
| | Sy5BC2F2AG3002-910 |
| | Sy5BC2F2AG3002-934 |
| | Sy5BC2F2AG3002-1013 |
| | Sy5BC2F2AG3002-1028 |
| | Sy5BC2F2AG3002-1059 |
| | Sy5BC2F2AG3002-1063 |
| | Sy5BC2F2AG23701-1704 |
| | Sy5BC2F2AG23701-1728 |
| | Sy5BC2F2AG23701-1765 |
| | Sy5BC2F2AG23701-1819 |
| | Sy5BC2F2AG23701-1841 |

The yield field trial plots are laid out in a random split block design with a single replication, where blocks represent early, mid and late maturity groups to facilitate harvest. There are two-row 16-ft. plots, with the adapted parent, as a border row on each side. Seeding rate is eight seeds per foot. Cultural practices such as herbicide applications and fertilization are carried out following the recommendations for soybean. For example, Lasso (Monsanto, St. Louis, Mo.) is applied as pre-emergence herbicide at the rate of 3 qt/Acre and Fusilade is applied as post-emergence at the rate of 16 oz/Acre. At harvest, only the test rows are harvested and seed yield is adjusted to 13% moisture content to get the dry yield for each line using the formula: Dry yield=Actual yield×(1−% moisture at harvest)/(1−0.13). Seed yield per plot is converted into yield in bushels per acre using the formula: Plot size/Acre=lb/Acre. For example, yield measured in lbs. from a 16-ft×5 ft plot is converted to bushels per acre by multiplying it with a factor of 9.075. In all cases, the average percent yield increase of the plants carrying the Sy5 yield QTL derived from PI290136 is statistically significant (Analysis of Variance) higher than that of the plants homozygous for the adapted alleles (Table 4a and 4b).

TABLE 4a
First year field test mean yield of Sy5 yield QTL

| Genotype | Mean (bu/Ac)² | N³ | Duncan Multiple range¹ |
|---|---|---|---|
| Homozygous Sy5 QTL | 54.35 | 4 | A |
| Heterozygous Sy5 QTL | 53.47 | 4 | AB |
| Sy5 QTL negative | 44.23 | 4 | BC |

¹SAS grouping of statistically significant populations.
²Yield is measured as dry seed weight in bushels per acre.
³N is the number of replications

TABLE 4b
Second year field test mean yield of Sy5 yield QTL

| Genotype | Mean (bu/Ac)² | N³ | Duncan Multiple range¹ |
|---|---|---|---|
| Homozygous Sy5 QTL | 38.25 | 4 | AB |
| Heterozygous Sy5 QTL | 41.30 | 4 | A |
| Sy5 QTL negative | 31.69 | 4 | B |

¹SAS grouping of statistically significant populations.
²Yield is measured as dry seed weight in bushels per acre.
³N is the number of replications

DNA marker analysis is performed among the BC₂F₁ plants. Leaf tissue is collected and DNA extracted from each of the BC₂F₁ plants. Each line is genotyped with the same two SSR markers flanking the Sy5 locus that are used in the BC₁F₁ analysis (Table 1).

To facilitate the use of this exotic locus in improving yield of commercial cultivars the following procedure can be used. Briefly, a cross can be made with any of the progenies derived from the above described plants and derivatives thereof carrying the exotic Sy5 yield locus with any potential cultivar that one wishes to improve. Using molecular marker analysis described earlier, one can monitor the positive transfer of the exotic yield-enhancing locus by checking the presence of the molecular marker band corresponding to the SSR markers. Then a series of backcrosses (up to BC₅) to the commercial cultivar (recurrent parent) can be made to recover most of the agronomic properties of the recurrent parent. Prior to each backcross step, the positive transfer of the exotic alleles has to be validated among backcross-derived progenies (BCnFn) (where n=generation) using molecular marker analysis as previously described. The number of backcrosses depends on the level of recurrent parent recovery which can also be facilitated by the use of markers evenly distributed throughout the genome.

Besides increased yield, other phenotypic expressions of the yield QTL from PI290136 can be observed. Increase in Glycine max plant height is a phenotypic marker of the QTL as shown in Table 5. When the Glycine max geneotype is homozygous for the QTL there is a significant (CV=5) increase in plant height. The mean values shown in Table 5 are the averages of the height of the main stem of five plants in two replications of field grown plants. Plant height is a component of yield for soybean.

TABLE 5
Comparison of Soybean Plant Height (cm) at Maturity

| QTL Genotype | N | Mean* |
|---|---|---|
| Homozygous Sy5 QTL | 48 | 42.18^{A} |
| Heterozygous Sy5 QTL | 48 | 40.78^{A} |
| Sy5 QTL negative | 48 | 33.05^{B} |

*values with the same letters are not statistically significant (Duncan's multiple range test)

### EXAMPLE 3

The genetic linkage of marker molecules of the present invention can be established on soybean linkage group U03 by a gene mapping model such as, without limitation, the flanking marker model reported by Lander and Botstein,Genetics, 121:185-199 (1989), and the interval mapping, based on maximum likelihood methods described by Lander and Botstein, Genetics, 121:185-199 (1989), and implemented in the software package MAPMAKER/QTL (Lincoln and Lander, Mapping Genes Controlling Quantitative Traits Using MAPMAKER/QTL, Whitehead Institute for Biomedical Research, Mass., (1990). Additional software includes Qgene, Version 2.23 (1996), Department of Plant Breeding and Biometry, 266 Emerson Hall, Cornell University, Ithaca, N.Y. Use of Qgene software is one such approach.

TABLE 6
Genetic linkage of molecular markers on U03 associated with Sy5

| Markers | Distance |
|---|---|
| 1 Satt315 | 6.9 cM |
| 2 Sy36 | 0.6 cM |
| XET1 | 0.3 cM |
| 3 SCNB187 | 0.1 cM |
| SAHH | 0.1 cM |
| 4 SCNB188 | 0.1 cM chalcone synthase gene cluster |
| 5 SCNB190 | 0.1 cM |
| 6 Sy5O | 0.0 cM chalcone synthase gene cluster |
| 7 Seedcoat color | 1.1 cM |
| 8 Sat_212 | 0.4 cM |
| 9 Sattl87 | 0.1 cM |
| 10 Sat_215 | — |
| | 9.8 cM |

Soybean gene sequences found on U03 to be in genetic linkage with the Sy5 locus comprise the S-adenosyl-L-homocystein hydrolase (SAHH) gene (SEQ ID NO:26), xyloglucan endotransglycosylase (XET1) gene (SEQ ID NO:27), and the chalcone synthase gene cluster (SEQ ID NO:28-37). Sequences derived from these genes can be used as molecular markers to track the genetic region containing the Sy5 locus.

### EXAMPLE 4

A BAC Library is constructed from SyS QTL containing soybean plant tissue. The single copy BAC vector, pBeloBAC11, is obtained from Dr. Hiroaki Shizuya (Shizuya et al., 1992) and prepared as described by Woo et al. (1994). Megabase soybean DNA embedded in agarose plugs is obtained as described by Zhang et al. (1996) using young greenhouse grown leaves from the Sy5 soybean plant ATCC #PTA-2323 or Glycine max PI290136 plant. Partial digests of megabase DNA are performed as follows: chopped plugs are distributed in 100 μl aliquots and incubated on ice for 30 minutes with 14 μl 10× enzyme buffer, 14 μl 40 mM spermidine, and 1.4 μl BSA. After a second 30 minutes incubation with 2 units HindlIl on ice, digestion reactions are allowed to proceed at 37° C. for 30 minutes. Digestions are stopped by placing on ice and adding 1/10 volume 0.5 M EDTA. Partially digested megabase DNA is subjected to two size selections by pulsed field electrophoresis (CHEF mapper apparatus, BIO-RAD). Initial size selection conditions are; 1% low gelling temperature agarose, 1-50 sec linear ramp, 6 volts/cm, 12° C., 22 hour run time, and 0.5× TBE buffer. Two fractions between 120 and 350 kb are cut from the gel based on a 50 kb lambda ladder reference (New England Biolabs, Beverly, Mass.). Gel slices are transferred to a second CHEF of similar composition and run at a constant 4 seconds switch time under similar time and temperature conditions. Two gel slices are excised and DNA is removed from the agarose by electroelution using the BIO-RAD Electro-Eluter (Model No. 422) system. Ligations are performed in 150 μl reactions using 30 ng vector and 300 ng DNA and allowed to proceed for 16 hour at 16° C. After desalting ligations, transformations are performed using 2 μl ligation reaction and 20 μl competent cells (DH10B, Gibco/BRL). Electroporations are performed on a cell porator with voltage booster (Gibco/BRL) using 320 volts at a resistance of 4 KW. Transformed cells are diluted immediately with 0.5 ml SOC (Sambrook et al., 1989) and incubated at 37° C. for 60 minutes before being plated on selective medium (LB, Luria-Bertani medium) with 12.5 μg/μl chloramphenicol, 0.55 mM IPTG, and 80 μg/ml X-Gal. After a 20 hour incubation at 37° C., the plates are placed at room temperature in the dark for an additional 20 hour to allow stronger color development of nonrecombinant colonies. After determining insert sizes of clones, ligations derived from the 225 to 300 kb gel fraction are utilized for additional transformations to construct the library. Recombinant white colonies are picked robotically (Genetix Q-bot) and stored individually in 538384-well microtiter plates (Genetix) containing 50 μl freezing broth (Woo et al., 1994). After incubation overnight, microtiter plates are stored at −80° C. Three copies of the library are made and stored in separate −80° C. freezers.

To prepare BAC DNA for clone characterization, 3 ml LB chloramphenicol (12.5 μg/μl) cultures are grown overnight in 6-cell autogen tubes and miniprepped robotically (Autogen 740 plasmid isolation system). To estimate insert size and determine distribution of clone size, BAC preps are performed from clones selected at random throughout the library. The BAC DNA is digested with 7.5 units (10 hour at 37° C.) of Not I restriction endonuclease (New England Biolabs) and analyzed by pulsed field electrophoresis in 1% agarose gels (6 v/cm, 5-15 sec switch time, 15 h run time, 14° C.).

To determine the size distribution of BAC clones in the library, the BACs are analyzed with Not I digests are grouped by insert size and the frequency of each group of clones represented in the library is determined. Based on this analysis, 95% of the clones in the library should have an average insert size equal to or greater than 100 kb. Of the clones larger than 100 kb, 67% should be equal to or greater than 125 kb.

The BAC inserts are probed by hybridization with the SSR marker DNA molecules and probes to the gene sequences to select BACs that form a contig including the Satt187, Sat_{—}212, Sy50, chalcone synthase genes sequences, SCNB190, SCNB188, SAHH, SCNB187, XET1, Sy36, and Satt315. There are at least two major methods for identifying BAC clones harboring molecular markers; hybridization to high-density BAC arrays using radioactively labeled probes, and PCR screening of pooled BAC DNA using primers designed from mapped marker sequences. While both methods are based on DNA sequence homology, the former is based on hybridization to the entire probe, and the latter employs primer annealing and subsequent amplification.

For PCR screening, if stringent primer design and PCR conditions are employed, only the BAC clones encompassing the marker sequences are identified. In contrast, BACs harboring sequences that are related, but not identical to the marker sequence, are identified when the BAC libraries are screened by hybridization. In general, PCR screening is more discriminating than hybridization and fewer candidates containing members of gene families and psuedogenes are identified in the screens. On the other hand, screening by hybridization readily permits the use of multiplexed probes, facilitating parallel processing of large numbers of markers. In addition, the generation of pools for PCR screening is labor intensive and only a limited number of pools can be processed in a given day. However, once the pools have been generated and the DNA prepared, there is sufficient DNA available to screen the library with thousands of different primer pairs.

To create working stocks of BAC DNA super-pools and BAC DNA sub-pools for SSR/PCR screening the primers and amplification conditions are selected to permit primer sequence length to be 15-40 nucleotides in length, preferably 20-25 nucleotides, and even more preferably 25-30 nucleotides in length. PCR conditions are based on the product length, ranging from 100- to 250-bp, or 250- to 500-bp, or 500- to 4000-bp. For a 20 μl reaction, 20 ng of genomic DNA is used. Initial PCR screening is carried out using genomic DNA (Sy5 line, 20 ng) as a template, as well as a template from a different plant species (Arabidopsis) as a negative control. The reactions are done in a thermocycler following manufacturers' set up conditions. The BAC library from the soybean line containing Sy5 should generate a PCR product which is identical to that amplified from the genomic DNA template. Negative controls, Arabidopsis genomic DNA and dH₂O, need to be included to ensure that the observed PCR products are not due to contamination or non-template dependent amplification. If the PCR conditions are not optimal, there will either be no DNA band or faint DNA bands will be observed. For BAC pool screening, optimal conditions should produce a strong single band of the predicted size. Once optimal PCR conditions are established, the same conditions will be employed for subsequent screening of the BAC pools.

Following establishment of the PCR conditions, the super-pools are screened. This can be accomplished by using the super-pooled DNA as template and the appropriate primers and PCR conditions that have been optimized for genomic DNA. When screening the super-pools, it is essential to include a reaction containing genomic DNA (positive control) and a reaction containing water (negative control). When positives are observed, the observed band must co-migrate with the cognate band amplified from the genomic template (Sy5 line). Positives that show up on the 2% agarose gel should be considered potential candidate pools. Once positive candidates have been identified, the corresponding sub-pools are screened to identify the BAC clones containing the marker of interest.

Screening sub-pools by PCR identify the clone candidate for that particular marker. The PCR conditions and the master mix formula developed for the super-pool screening are used for screening the respective sub-pools. Optimal screening of the sub-pools should yield a single reaction in each dimension of the sub-pool.

BACs are identified by hybridization to high-density BAC library membranes using marker DNA sequences, such as ESTs, STSs, RFLPs, AFLPs, SSRs and RAPDs. Sequences of the present invention are used to screen the BAC library membranes. In order to positionally clone the Sy5 yield QTL, the BACs are identified that comprise the markers described herein. Based on the identified BACs, chromosomal walking methods are performed that identify adjacent BACs to construct contigs that cover the region of the Sy5 locus. There are several methods to accomplish this task, including the BAC pooling and the PCR screening method, also hybridization methods offer a quick and efficient means of localizing markers to BAC clones.

The general process for identification of BACs by hybridization includes following procedures: (I) Purification of probes—the probes used for hybridization are usually derived from clones or genomic DNA by either PCR amplification using the vector or gene-specific primers, or digestion of cloned DNA using restriction enzymes. As probes containing any vector or repetitive DNA sequences will cause a high background, isolated DNA fragments may be gel-purified before labeling; (II) First round hybridization to high-density BAC library membranes—in the first round hybridization procedure, multiple probes are labeled separately, then pooled together to hybridize to BAC filters. Positive BACs identified in this procedure are deconvoluted by rehybridization with the individual probes. As some markers have a limited length of non-repetitive DNA sequences, like STS or SSR markers, two hybridization methods are used as a preferred method to identify positive BACs corresponding to marker sequences: random priming labeling and hybridization and the Overgo oligonucleotide labeling and hybridization method. Random priming labeling method is recommended for probes longer than 100 bp, whereas the Overgo oligonucleotide labeling method is used for probes shorter than 50 bp, especially for SSR and STS markers. Combine labelled probes in one tube and denature@95° C. for 3 min. and add directly to 10 mls of prewarmed (58° C.) HyperHyb (Research Genetics) solution. Add equal amounts of the probe/HyperHyb solution to each bottle. Avoid adding directly on the membrane. Incubate for 1.5-2 hours at 58° C. in 70 mm bottles in a rotating incubator (10 RPM). Wash filters by adding 100 mls of prewarmed (58° C.) 1× SSC,0.1% SDS to each bottle and return to rotating incubator for 15 minutes., repeat two additional times. Remove filters from the bottles and combine into a tub filled with 2 liters of prewarmed (58° C.) 0.1× SSC,0.1% SDS. wash for 15 minutes. on rotating platform. Expose filters to x-ray film for 4-24 hours, develop film and identify positive BACs on autoradiograph. Pick identified BACs from the original plates and isolate DNA. Spot isolated DNA onto another membrane and repeat procedure as described above.

The DNA is isolated from the rescreened BACs and is then sequenced. The sequence is compared to DNA sequence from the same genomic region isolated from soybean not containing the enhanced Sy5 yield QTL. The polymorphisms between the DNA sequences are used to identify DNA regions that may contain the QTL. These regions inserted into plant transformation vectors and then are transformed into plants not containing the QTL, the plants are regenerated, then screened for the enhanced yield effect. Those plants with enhanced yield contain the isolated QTL.

Yellow seed coat Glycine max sibling plants from the progeny of BC₂F₄ plants that are selfed were deposited with the American Type Culture Collection (ATCC, 10801 University Blvd, Manassas, Va., U.S.A., 20110-2209) on Aug. 2, 2000 and assigned ATCC No. PTA-2323.

Having illustrated and described the principles of the present invention, it should be apparent to persons skilled in the art that the invention can be modified in arrangement and detail without departing from such principles. We claim all modifications that are within the spirit and scope of the appended claims.

All publications and published patent documents cited in this specification are incorporated herein by reference to the same extent as if each individual publication or patent application was specifically and individually indicated to be incorporated by reference.

We claim:

1. A method of soybean breeding for a yellow seed coat Glycine max plant having enhanced yield comprising:
(A) crossing a black seed coat Glycine max PI290136 parent plant or progeny thereof with a yellow seed coat Glycine max parent plant to produce a segregating population of progeny plants; and
(B) screening the segregating population of progeny plants for the presence of a DNA molecular marker of a sufficient length that is homologous or complementary to a DNA molecule selected from the group consisting of SEQ ID NO: 19-37, wherein a member of the progeny plants has an enhanced yield allele derived from the Glycine max PI290136 plant and that maps to linkage group U03 of the Glycine max PI290136 plant; and
(C) selecting the member plant for further crossing and selection, wherein the member plant selected has a yellow seed coat and enhanced yield relative to the yellow seed coat Glycine max parent plant.

2. In the method of soybean breeding of claim 1, wherein Step B further comprises: screening the segregating population for the presence of a DNA molecular marker with a DNA primer pair selected from the group consisting of SEQ ID NO: 1-18 primer pairs; and performing a DNA amplification method.

3. In the method of soybean breeding of claim 1, a DNA molecular marker of sufficient length is about 11 contiguous nucleotides homologous or complementary to a DNA molecule selected from the group consisting of SEQ ID NO: 19-37.

4. In the method of soybean breeding of claim 1, a DNA molecular marker of sufficient length is about 18 to 24 contiguous nucleotides homologous or complementary to a DNA molecule selected from the group consisting of SEQ ID NO: 19-37.

5. In the method of soybean breeding of claim 1, a DNA molecular marker of sufficient length is greater than 24 contiguous nucleotides homologous or complementary to a DNA molecule selected from the group consisting of SEQ ID NO: 19-37.

6. In the method of soybean breeding of claim 1, wherein said yellow seed coat Glycine max parent plant has an agronomic trait selected from the group consisting of herbicide tolerance, increased yield, insect control, fungal disease resistance, virus resistance, nematode resistance, bacterial disease resistance, mycoplasma disease resistance, modified oils production, high oil production, high protein production, germination and seedling growth control, enhanced animal and human nutrition, low raffinose, environmental stress resistance, increased digestibility, industrial enzyme production, pharmaceutical peptide and small molecule production, improved processing traits, proteins improved flavor, nitrogen fixation, hybrid seed production, reduced allergenicity, biopolymers, and biofuel production.

7. In the method of soybean breeding of claim 1, said enhanced yield is at least 2 percent greater than the yellow seed coat Glycine max parent plant.

8. In the method of soybean breeding of claim 1, said enhanced yield is between 5 and 10 percent greater than the yellow seed coat Glycine max parent plant.

9. In the method of soybean breeding of claim 1, said enhanced yield is greater than 10 percent yield increase of the yellow seed coat Glycine max parent plant.

10. A method of soybean breeding comprising production of a yellow seed coat Glycine max plant having enhanced yield comprising:
(A) crossing a Glycine max plant provided by growing a plant from the seed of ATCC deposit #PTA-2323 or progeny thereof with an elite Glycine max parent plant cultivar to produce a segregating population of progeny plants; and
(B) screening the segregating population of progeny plants for the presence of a DNA molecular marker of a sufficient length that is homologous or complementary to a DNA molecule selected from the group consisting of SEQ ID NO: 19-37; and
(C) identifying a member of the segregating population f progeny plants that has the DNA molecular maker that maps to linkage group U03 of the Glycine max plant ATCC deposit #PTA-2323; and
(D) selecting the member for further crossing and selection, wherein the member selected has a yellow seed coat and enhanced yield relative to the elite Glycine max parent plant cultivar.

11. In the method of soybean breeding of claim 10, wherein Step B further comprises: screening the segregating population of progeny plants for the presence of a DNA molecular marker with a DNA primer pair selected from the group consisting of SEQ ID NO: 1-18 DNA primer pairs; and performing a DNA amplification method.

12. In the method of soybean breeding of claim 10, a DNA molecular marker of sufficient length is about 11 contiguous nucleotides homologous or complementary to a DNA molecule selected from the group consisting of SEQ ID NO:19-37.

13. In the method of soybean breeding of claim 10, a DNA molecular marker of sufficient length is about 18 to 24 contiguous nucleotides homologous or complementary to a DNA molecule selected from the group consisting of SEQ ID NO: 19-37.

14. In the method of soybean breeding of claim 10, a DNA molecular marker of sufficient length is greater than 24 contiguous nucleotides homologous or complementary to a DNA molecule selected from the group consisting of SEQ ID NO: 19-37.

15. In the method of soybean breeding of claim 10, wherein Step B comprises: screening the segregating population of progeny plants with a DNA molecular marker that is homologous or complementary to a DNA molecule selected from the group consisting of SEQ ID NO: 19-37.

16. In the method of soybean breeding of claim 10, said elite Glycine max parent plant has agronomic traits selected from the group consisting herbicide resistance, increased yield, insect control, fungal disease resistance, virus resistance, nematode resistance, bacterial disease resistance, mycoplasma disease resistance, modified oils production, high oil production, high protein production, germination and seedling growth control, enhanced animal and human nutrition, low raffinose, environmental stress resistance, increased digestibility, industrial enzyme production, pharmaceutical peptides and small molecule production, improved processing traits, proteins improved flavor, nitrogen fixation, hybrid seed production, reduced allergenicity, biopolymers, and biofuel production.

17. In the method of soybean breeding of claim 10, said enhanced yield is at least 2 percent greater than the elite Glycine max parent plant.

18. In the method of soybean breeding of claim 10, said enhanced yield is between 5 and 10 percent greater than the elite Glycine max parent plant.

19. In the method of soybean breeding of claim 10, said enhanced yield is greater than 10 percent yield increase of the elite Glycine max parent plant.

20. A DNA molecule associated with enhanced yield homologous or complementary to a DNA molecule selected from the group consisting SEQ ID NO:21-37 located on linkage group U03 of Glycine max PI290136 or progeny thereof and is of a sufficient length to be useful as a DNA molecular marker for an allele of a quantitative trait locus, wherein the allele provides enhanced yield in a yellow seed coat Glycine max plant.

21. A DNA molecule associated with an enhanced yield homologous or complementary to a DNA molecule selected from the group consisting of SEQ ID NO:21-37 located on linkage group U03 of a yellow seed coat Glycine max provided by ATCC deposit No. PTA-2323 or progeny thereof and is of a sufficient length to be useful as a DNA molecular marker for an allele of a quantitative trait locus, wherein the allele provides enhanced yield in a yellow seed coat Glycine max plant.

22. A method of providing an isolated DNA molecule containing an allele of an enhanced yield QTL comprising:
(A) constructing a soybean genomic DNA library selected from the group consisting of Glycine max PI290136 and Glycine max having ATCC Accession No. PTA-2323 containing the enhanced yield QTL phenotype; and
(B) hybridizing the soybean genomic DNA library with a DNA sequence selected from the group consisting of SEQ ID NO: 19-37; and
(C) isolating the soybean genomic DNA that hybridizes to the DNA sequence; and
(D) sequencing the isolated soybean genomic DNA containing the enhanced yield QTL and constructing a contig of sequences; and
(E) comparing the contig to a soybean genomic DNA sequence not containing the QTL; and
(F) identifing the polymorphisms in the contig genomic DNA; and
(G) constructing a plant transformation vector containing the soybean genomic DNA with the identified polymorphisms; and
(H) transforming plant cells with the plant transformation vector; and
(I) regenerating the plant cells into plants; and
(J) selecting said plants for the enhanced yield phenotype.

23. A Glycine max plant comprising an allele of a quantitative trait locus located on linkage group U03 associated with enhanced yield in the Glycine max plant, wherein said Glycine max plant has a yellow seed coat and wherein said allele of the quantitative trait locus is also located on linkage group U03 of a black seed coat Glycine max PI290136 plant and linked to a DNA molecular marker selected from the group consisting of SEQ ID NO: 19-37.

24. A soybean seed having ATCC Accession No. PTA-2323.

25. A soybean plant or its parts produced by growing the seed of claim 24.

26. The part of the soybean plant of claim 25 comprising pollen.

27. The part of the soybean plant of claim 25 comprising an ovule.

28. A soybean plant of claim 25, wherein the soybean plant produces yellow seed coat seeds and is high yielding.

29. A soybean plant, or its parts, wherein at least one ancestor of the soybean plant is the soybean plant, or its parts, of claim 24.


SEQUENCE LISTING (37 sequences)

SEQ ID NO: 1 - DNA, 24 bp, Glycine max
gcgcgacaactctaatgaaaatct

SEQ ID NO: 2 - DNA, 23 bp, Glycine max
gcggagtttgatttttcaaaagt

SEQ ID NO: 3 - DNA, 25 bp, Glycine max
gcgttttaatttatgatataaccaa

SEQ ID NO: 4 - DNA, 24 bp, Glycine max
gcgttttatctctttttccacaac

SEQ ID NO: 5 - DNA, 25 bp, Glycine max
atcaatcgacgcaataatcaagaaa

SEQ ID NO: 6 - DNA, 25 bp, Glycine max
atgatgagaagacaatgggatgtca

SEQ ID NO: 7 - DNA, 25 bp, Glycine max
caggcttcagtgtgcataatacagg

SEQ ID NO: 8 - DNA, 25 bp, Glycine max
ttctatgttccctgtgcaaacactg

SEQ ID NO: 9 - DNA, 25 bp, Glycine max
gtctgcaagctaacagtgtcagagg

SEQ ID NO: 10 - DNA, 26 bp, Glycine max
cacactcaatctcattagcagacacg

SEQ ID NO: 11 - DNA, 25 bp, Glycine max
tcctttggctcactattgacgattt

SEQ ID NO: 12 - DNA, 25 bp, Glycine max
acccgtgtgccactttaactacatt

SEQ ID NO: 13 - DNA, 25 bp, Glycine max
taacgctgcatgatttgagttctgt

SEQ ID NO: 14 - DNA, 25 bp, Glycine max
gtattggttggactttggagaccac

SEQ ID NO: 15 - DNA, 28 bp, Glycine max
gcggacaattttttatcaataatttatt

SEQ ID NO: 16 - DNA, 28 bp, Glycine max
gcgatgcttacttttcctatgatcactt

SEQ ID NO: 17 - DNA, 24 bp, Glycine max
gcgtagcaacaaagcaatctacag

SEQ ID NO: 18 - DNA, 29 bp, Glycine max
gcgtcccattttattccacactatgtaat

SEQ ID NO: 19 - DNA, 235 bp, Glycine max
cgacaactctaatgaaaatctttattattattattattattattattattattattattcacgaagttcccttaaaaaatctttagtaagacacatgcattaattatatgacaataaaaaaaaaaagaattcaaatgtttcaaaatgaaaaatcattaattcacttttatgtcaattattattattattattataacattaattactttgaattgacttttgaaaaatcaaactc

SEQ ID NO: 20 - DNA, 272 bp, Glycine max
ttttaatttatgatataaccaaatagtattcctattattattattattattattattattattattattattattattattattattaaaagttatacatgtaaatatttttttaaggtgacattctgaataaatttttatatgtgatttgggaaaagtagagacaagttcaccctaaaattaatattcagtaagtggaacgtctccaaatttattataaaaattgtaaatatttattctatgcgactgaagttgtggaaaaagagataaaa

SEQ ID NO: 21 - DNA, 280 bp, Glycine max
atcaatcgacgcaataatcaagaaaatcaaacatggtatcagtaattaattttaaataagattatatatatatatatatatatatatatatatatatatatatatatatagacaccccaataaaaatcatattaaaacaattataattcataatattcagaataaataaaaatattgaaataaatggcaacacctcatcgtattcaaataaatataattgacacaactttatactcaattttttggttcctggaatgacatcccattgtcttctcatcat

SEQ ID NO: 22 - DNA, 366 bp, Glycine max
caggcttcagtgtgcataatacaggtttctgttggtgggactttctcccaacatttcattttgggattttctcccaacctttattttgtctgaccttagtcgtaatagttctaaccttccttccttccttcatgtttcattcgtgatcctgttttttggtatttcagggggttgtttgagcctagtagggggccaggtgtcaacctatagttgggatttcaccccttaggctgaaatttcctttcctcacttaagtaaaaaaaaaaacaaaaagttttagtttttgtatgaaaatgcttttttatagcaattttatatgattagaaaattaaactattccccagtgtttgcacagggaacatagaa

SEQ ID NO: 23 - DNA, 96 bp, Glycine max
gtctgcaagctaacagtgtcagaggatatgaatattagtattattaacaataataataataatgatgaaacgtgtctgctaatgagattgagtgtg

SEQ ID NO: 24 - DNA, 321 bp, Glycine max
tcctttggctcactattgacgattttctcgatgattaattgacccaacattctgtttgtaactttatttataaaacaaatatttgtacttcaattataacaacaaatttaagaagaatatatatatatatatatttgtgatggaaatgatcatgaaagaaacagaatcaatatttcttataatcaagaaaaataatagactcatttatttcttataaaaagaaggagataaagtataaaatacaaatggtaaacataaaagaaaaaaaaactttttttgaccggtatggtaacgaaaatgtagttaaagtggcacacgggt

SEQ ID NO: 25 - DNA, 185 bp, Glycine max
taacgctgcatgatttgagttctgttttgtcggcggggactagggacaaatatattttttgttagttaatttgtatatttattggtgatatgtctgaagttaagttaattggccatgcatgtgtgtgtgtgtggtagtgagaagaattgagaaaaagaatgtggtctccaaagtccaaccaatac

SEQ ID NO: 26 - DNA, 3830 bp, Glycine max
tgtgttttacaatatttagagaaacttggttgatatcacaaaaaattgtaagacaaaattaatgtcaagtgagtttagaatactaaatgaaaattttaacataaaaaaaaaaaaatcaatggaatggaacccatccagcgcaactagctgagtcacatacagtgccaaaagacatgggtactacaaatgctcactttagtggctatggaacaaccatcagcattcagctcttccttttttctgtcgtaggccaagagacaaagtttgtcacaggtttacaaattgattgtggccacaatcacacggtaaacattagaatggaagaaaaaaaatctgtctatgatcgatgtcgtgaacttcacccactccatcaatgaagaatttattttaaatacagttacacaccaacttaataagactttttgcacaaaattacctgattgggaggaatatgaattgtcttataaatcacgtattcacaagttctacttttacaaaactctttacatgtattttccaaaaaaagaaaaatctttacatgtatgttaacctacctaacaaatctctaattaacctataaattttttaaatgctttttgagaaaactttataggcagatagaagattgttgagagttttttaaatgcttatcaacaatctccgatagtcccttagctttaccaagtacatgaaaatcttacatataatgcttttactttaccaactattaacttgagcaccgaaatctttaccagtatgctcatttgatgcatattaaaatgtacaaaattttatagaggcctgatcaataccatcgaatgaaaccttaatgacatgctacttgttagcgatgtcaataaaggcttactcaaggattattccacaggcctaaatcatagacaattttacttaattgtatttattcaattagtccttagatgtcaaagaatctattagatgatagttttagtggcatgatagagaatgaaacccacatctataaaaaaaagaagacaaaagttagttttagatctttaatcacttgtgtgaattcatattagttttacgtgtattcgaagtgaaaatattcatctgtatgagaccataaacattcttatgagagacttgtttgaagtataatttttcatagtacagtaaagctgattgttgttttttctcgtacgcaaaatttatattcaggacaatgtttaagagtgaaaacataataaaattaacctcacaaaaagtaagtatatatatatatatatatatatatatatatatataaatctcaatcaattaaaataataataaggacaaataaatagattctcacaaaatataatttattattaaattaatttttaacattataacttaacgataaaatattttttttatatttttttatgaactaatttaacaactcatcacatcttgcaaaacaaaatgaatcatttatcctaataataatttaatttaggcgtttattttatgatgatttagcatctttttgggagaatactaaaaaacatataaaagaaaaagaaatattcaggatgaaaaatgaaatgcgtgtgaaaattggaaggaggtaaggctgggtcgacccagatctagttgagctcaccaactcccgctcccatttccttatttatagacagagtctgattgtttcctcaccactccctccactctctttctctagtcctgttatttctcagcgcgtaaagcatggctttgttggtggagaaaaccacgagtggtcgcgagtacaaggtcaaggacctttcccaggccgacttcggccgcctcgagatcgagctggccgaggttgagatgcccggcctcatggcctgtcggaccgagttcggcccctcccagcccttcaagggggcccgcatcaccggctccctccacatgaccatccagaccgccgttctcattgagaccctcaccgcccttggcgccgaggtccgctggtgctcctgcaacatcttctccacccaggaccacgccgccgccgctattgcccgcgacagtgccgccgtcttcgcctggaagggtgagaccctccaggagtactggtggtgcaccgagcgcgccctcgactggggccccggtggtggacccgacctcatcgtcgacgacggtggtgacgctacccttctcatccacgaaggcgtcaaggccgaggagctctatgagaagaccggcgaactccccgaccccaactccaccgacaacgccgagtttcagatcgtgcttaccatcatcagagatgggttgaagaccgatcccaccaggtaccgcaagatgaaggagcgtctcgttggggtttctgaggaaaccaccactggagttaagaggctctatcagatgcaggcgaatgggactcttctcttccctgctattaatgtcaatgactctgtcaccaagagcaaggtaatgtctctttttcccccagatctagtgtcttttttgtgttaaaatgtaggattgagttcggatctgttgtttttggatgggttttgtgccattggtgaaatgaggttttgaacctgtcaactgtttgactaatgtcctctaagaagtctggatcggtattgggtgctattttagtgtgtttggatctgtgtgttgaaacgtcagaacattagtaagttgcttgctaacgtgactttaggtaaatggtcacatgttttattacacaaataaggaattgattctgagtgcacattttgatttgaagctacttttggataggataaaataaattatactgaattttactactgtttttggttttaaaataaaaaaatgttcaaacataaatcatgttgtttcaaaatcaattttaactcgaaatcgttttcattcaaaattggttttgcaaacattgatccaaaccgagtcttttgtgacgggttgtttattgattagggtattgaaagtaagaagtgggtgattggattttgaggacattatactagctggtcatggatctagttgattataattggattttgctttgttgcttgtgttttgtttgtttaaccttttaatctgtggttttgtaacagtttgacaacttgtatgggtgccgtcactctctccctgatggtctcatgagggctaccgatgttatgattgctggaaaggtggctgttgtggctggatatggtgatgttggcaagggttgtgctgctgcaatgaagcaggctggtgctcgtgtcatcgtgaccgagattgatcccatctgtgcccttcaggctctcatggaaggccttcaggttctgaccttggaggatgttgtttctgaggctgatatctttgtcaccaccaccggtaacaaggacatcatcatggttgaccacatgaggaaaatgaagaacaatgccattgtttgcaacattggtcactttgacaatgagatcgacatgcttgggctggagaactaccccggcgtgaagcgcatcaccatcaagccccaaactgacagatgggtcttccctgagaccaacaccggtatcattgtcttggctgagggtcgattgatgaacttgggatgcgccactggacaccccagttttgtgatgtcctgctccttcaccaaccaggtcattgctcagcttgagttgtggaaggagaagagtaccggcaagtacgagaagaaggtttacgttttgcccaagcaccttgatgagaaggtggctgcacttcacctgggcaaacttggagctaagctgacccagcttagcaagtcccaggctgattacatcagtgtgcctgttgagggtccatacaagcctgctcactacaggtactaa

SEQ ID NO: 27 - DNA, 4096 bp, Glycine max
agtgaaggacactaattaaattccctcaaccatacatattcacattaaaatcaggtcccttctgaggtgctgtatacattctcacattcattaaaatagtactttttaaataaggcatcatcattttaattactttttgcaagaaaaggttggagattctgctagctggttgccataagttgattcccactgaccatctccttataagttataaccaataaatttgcacttttattctaataattaactagttagtggtggttaattaacattagagggatggaaggctacacttcaatgatgatttgcactaatgaatagtagtttttaagcatccaaatactccaactcttgagttttgatctagtttctaaatgttctaataattatattataatttgtaacacttagcggtacatactttagtgatgaagtgatcattcattgccatactcttcgttactgtgccattgtggatacccttaccctcatttcaaggttgattcttgtagaacttccttattaaatgctttggaccatttatcaggaaaaaaagtaatctgtggctattgtaacattggagggtgggtgcaggtaggaagtttgttcatttactaataatttttctcattaataatctgtcatacaagtagattttaatataattgtatatgcgccgtactcgtgagaaataaatgcatattggtttgaattattatttttatttgttttgtcatgcaaatccaaagttgttgtctgcattggaaaagacaaattaaaactcaagcaatacaacaacccgagacaaagcaagcaggaaaagagttatcagcatggccgaagtggataaccatgccatatcattggcaatctcgtgactatttttttgaattttaactccaacatcaaagaatatctatatctatatgtcataaaattgaaaattaacagtgaaagtttaggcgatggtttaggcaatagcataggggcaataacgcaggtacgaactctgccacatggcatcatctaagtggatccataattcatgattggtggtactaagaagtggtaaaataccctcacgtctttattctccttccacatcacacccagttggcatccatccatcacctaattttctcttttttttgaaaaaaaaagggatattttgttccaaatcatacaaaaatggggtctacccctacatttcaggtataaaattctctttttttttatcattacttttttatttgtgagcaatatcatgtacgcaatcattgttcatacttcatattactactaaaacttaaggttcaggtgcgttgatacgagagaaaataatttatttaaaaaaaaattatgtttgattttcgttatgtgtaaaatttctttgagttgataattacatatcacaaacaaaattaatttctaatctaatgattaaaagaaactcggaatctggaatttgtgactcaggacaaagatactactactgaataagtgaatagcatcctgtgcacaaacccaaaaaacatcacaaaatccatttaagtataaccaatgcccaaacaaaaaggttccagctttcaaaacttgctaagctggcaccagcttttggtcccaccagcccaagttattgctccttcacgcgtccaaccatagtcccatacccaaatcccatcttccatttctctctttttcacacatatatatatacccctcttttgaacacattccctcacatcatcacaagaagcacaatttctctttctctctttttttgtgtgtccaaaatggctcctagttctgctcacaacaatgggttctatgtgcttatgctagttgggatagtggttagcactatggttgctacctgtgctggtagcttctaccaagactttgatctaacatggggtggtgaccgtgctaagatattcaatggtggccagcttctatcactttccctagacaaagtctctggctctggcttcaaatcaaagaaagaatacctatttgggaggattgatatgcagctcaagctcgttgccggcaactctgctggcactgtcactgcttactacgtatgtttattaatatttacaataattatatatgtttgtacattattttcatcactacaatatataatctatgatacaaacaaatatttcaaacacaacttaatacaggtttcttagctacttgtagtatcaaaattacagtttcatctagataatttgcataatatataggtttctaataaatgtcaacatagatcactgagataaactctaattctcatcacaaaataaccccaagagtatgttttaatgaaatctacccttcccaaatttttttaaaaaagagagttaaaaatgctataaattttgtgaggtgcaattatcatgttatctgcttcatcttttttatttctggtatactcatttacccttgtttttaccatataacaaaactatactaattcaaattgattagtttctttccttctccatatatatatatatatatattatatatatatatatgagctaaaacagtaatactgtagagtttttgtatgtgtgtgtatgtttgtttttcttttaggtagttttagcattgattcttgatgaaagaacatgacttatcctgtcttcaaatacgaccactattgaccacttttacacttcaaacatcaacctttgtcaaactcaactgtacattcacgagaatgctattgtagcaaacccacaaaaacaagttagagtacagaattttactttgtcaacaactaatgctttatttattcattccatgctgctttctgtttcaaacattgacgtatttttttttatacaattcaaacattgacgtatacattaatcaacttggtctttttaaagcagtgaatttaacaagcgctcgtgacaggggaaggtggctaactttgacctagtccaaaacattaacaacttttaatattgaaaacttcggttcatagcataatctaatgacaaataaaaaaaaacgctctcatggtcgaaccttcacataaaaatacttttatcacaatgagttttctggttttgaattgataaaaaaaaaaaatctaagaccttgtttagttgctaaactcatactgttcctatgcatgcacactatttaaattactgttaataaacaacaaaaatgacaattccccaaaataaggtcattttcttaatttgtcgagttgtttgtgctgctaccacacacaaaggccatatcaataactatagtagtaattccattttctgcggtgcagttgtcatcccaagggccaacacatgatgagattgatttcgagtttttgggaaacctaagtggggacccttatattctccacacaaacatcttcacccaaggcaaaggcaacagggagcaacagttctatctctggttcgaccccaccagaaacttccacacttactctatcatttggaagccccagcacatcatgtaagtcacaataaacaaatattaaaaaaaatacacattttttttattagtaaatattctatacactaatactgcaaaagattttatatcaactatctttgaactataagtcataccatttgaaagtgtaaaaaatttacattgaaactggatagaaattaaactttgtttatctctatgcttttcaccaatatccatttaccaaatcatgaattgggttaactgcagattcttggttgataacacacccataagggtattcaagaatgctgaacctcttggtgttccttttccaaagaaccagcccatgagaatctattctagcctctggaatgctgatgactgggccaccagaggaggattggtgaaaactgattggtccaaagcaccctttacagcatactaccgcaatttcaaggccattgagttctcatccaagtcttccatttcaaattctggggctgaatatgaggcaaatgagcttgatgcttatagcagaagaagactgagatgggttcagaagtacttcatgatctataactactgcagtgatctcaagcgattcccacaaggtcttcctgctgaatgtaaacgttga

SEQ ID NO: 28 - DNA, 3086 bp, Glycine max
caatgatattttaaacctgtgacccactaattcacaaacatttaattgatataaattttaaataaaatattctcaatttattaactcattttgttataagctaattatcccattagccatcaataacaataaattttactattcatcgactattttttttatgataaatgtctcttttaattgcatgtgttaattgatctttttaattatgcttaagaatagtatttaaaaaatagtttaaaaagctaaaaagattattgttttgaaaaaaaatagaaagaccatttgttttaggaaggagggagtattatatgcaatagtctgtttatcattaaatgaatattaatttttgttacaattttttataagtcgtgttttttttactattttttaaatgaaaaatgaataatttaatacattctcaactttttttatatttagtttagtgtagtgaaattaagcacaatttcaccttttttttaaattgtttaaaattcacgactccgcattatattataatatattgtgttaatattattagtaaataattttttctcatttactatttggttgagagaataaggttatattattagcaaatgcattatttgacaaattttaattaagttcctaaattattttttttcaattgttctcttaacttatatttttttaaatgatgttcctaaactattaggaataaatgtatatgtccaagaatcaatctgtcatgtaactaattaggaataaatattattagaatttgatcatcatgtactactataaaacaattgattggataatatctttaattaaaatcatggactcattatcataaactagtattgtataaatttaatccaaattaatcttgattataaaaaacaagagacatccaaattcaaaaaataatagcatttattaaataaagattaataaatttcatttattaaattacacatatagatgatatatatgtgaatataattctaaaagttaataacattactttaaattatcaataaaaaattcataagaaaaaaaaaataattttgttttacttaaaattatcataataattaataagttctttattatattttaattttggacatcttctatctattttttaaacaagatacccaatatcttaaggtattagttgaatagttattaagtaatgactaatgagtctgagttttatttaaaacaattattttttcgaattatttttctgggcgataaatgaacttaaactaatcatttacgcacaatattaaaacaagtaaatctctcgtgacatttctttttgatacacttgaaactgatcaaaactaatttcttaccagggatatgagtccctttcattcacatcaacacacataacagtaagtaattatttttccaaaaactctaaccagaaataaaaaagtaattccaaaattaggagaagcaattgtaaagaagtatggactatggagaacaaaaaaaaaatttgctgattattgggggaaaagaatgggttggtgtgttgggagagtcaacagtctacttagacatgcggtacatacaccatatatttgaaagaaaaaaaagcgtagtcagaggaagcatgcgcgcatctacctacccacccttttcaattatgcatgtatatatatatctgagccactttgccacattcattcccaccctcatacccttttctttcgtgcctagctactccttaattactttcattctttaatttgctgcaagctatagcttcattagttcattcacaaaattaattattacaatggtgagtgttgaagagatccgtcaggcacaacgtgcagaaggccctgccactgtcatggctattggcaccgccactcctcccaactgcgtggatcagagtacctatcctgactattatttccgcatcaccaacagcgagcacatgaccgagctcaaagaaaaattcaaacgcatgtgtaagatatctctctcttttatcctatcttcatttcattatataatatgcatgttgcttatttccaacatatacctttgatttcattaatgatatcaatgaaatttaatttattatttcaggtgataagtcgatgattaagaagcgatacatgtacttaaacgaagagatcctgaaggagaatcccagtgtttgtgcatatatggcaccttcgttggatgcaaggcaagacatggtggttatggaggtaccaaagttgggaaaagaggctgcaactaaggcaatcaaggaatggggtcaacccaagtccaagattacccatctcatcttttgcaccactagtggtgtcgacatgcctggtgctgattatcagctcactaaactattaggccttcgtccctccgtcaagcgttacatgatgtaccaacaaggctgctttgccggtggcacggtgcttcgtttggccaaagacctcgctgaaaacaacaagggtgctcgcgtgcttgtcgtttgttctgagatcaccgcagtcacattccgcggcccaactgacacccatcttgatagccttgtgggtcaagccttgtttggagatggtgcagccgctgtcattgttggatcagaccccttaccagttgaaaagcctttgtttcagcttgtctggactgcccagacaatccttccagacagtgaaggggctattgatggacaccttcgcgaagttggtctcactttccatctcctcaaggatgttcctggactcatctccaagaatattgagaaggccttggttgaagccttccaacccttgggaatctccgattacaattctatcttctggattgcacaccctggtggacccgcaattttggaccaagtggaggctaagttaggcttgaagcctgaaaaaatggaagctactaggcatgtgctcagcgagtatggtaacatgtcaagtgcatgtgtgctattcatcttggatcaaatgcggaagaaatcaatagaaaatggacttggcacaaccggcgaaggccttgactggggtgtgctatttggtttcggtcctggactcactgttgagactgttgtactccgcagtgtcactgtctaa

SEQ ID NO: 29 - DNA, 3048 bp, Glycine max
tttttaatttttgacgaattttatcttaatctttaaattttggacattttatctcaacttttaataatcctacaaattttatccttcatcactttactagttacataattatattttttttatccctaacttattagtttttgccaaattttattccaactttaaatttttttgacaaaatttatccttaattttaattttttttgacaaattttaccccaacttttgtgcttataaatagataaataatagaggataaaattcacaagtttcttaaaaattgaaaataaaatgtgtcaaattaaaaaattagggataaaattcactaaaaattaaaaaattaaaaataaaaagtgcaattaagcctatgtgtaactacatacggtggaaaatcaaacatagattctcttgttaaataattaggtttgtatttaaaatgaaataacaacaaagtttattttctcaagaaaacaaaaaatgttcctaaaatttcctatgttgttattttagtatttaaatttaatttaactatattatattttaatttcgaaagtatgttattattgtcatttacatcgcatgacctttgaaactttggattaaaatgagttacctttggtcattttagcactttcaagactaaattaacagcgtcttacgcttttacttttacgaatttgttcacttatccgattaataaagacagatataaaaattaaaacccaacctaattcctgttgaatttaatttagtgagatcgagaaaacctttgggaaactttaaggatgattgggtcagcattttcatcgaatgcaatttgggaagcatcagtgtttggaatgggtttatgtgtgacaggttctgtggatttcacatcaacaataataataagcaatttttttcttctcaaaatcaaatttattcaattttggtattcggtggtgggaatacaaggcgttcaactggtgcttcatttggtttgctgatagcgataggtggttgcttttattttctcgtggttatgttctataatcggatggctgaattattcgtaaatgtttagaggctctgccaagttcagcaagataaagctatttttttcgtaattatgcaacatgttgctggtagatagctttgatgcacagcaaaattgtattctgatataactttcagtaggggcacaacttgtgcagctaagctgcttttaataatatttctatcctttgcatctcaagaaaaaaaaaattgttcattggattggagtcgattttagttttgccagaaataactgaatcaatccaaatcaaattgaattactaaatactattaacattaaagctactttgttgatgatgttgatacgatacactccctttttataatgtcaatgactatatcctttctctgtcaacaaatgactatgtccttttatccaaatctatttatttgagaatcattttaacgtgtttttaatcaaatttgtaaggtatatatataatcattataatgggatagtcaacagtcaacatagtcatgcagtgtacaatatagttgagagaaaacacagaacacagccaattcgttagaggaaacatgctcatcatctactcagtactcacctacccacttcaagttcaactgtctatctattcatatatatatacccacccttccaaaccactttgcaacatccatccaagccttttctttcctagctactacactttcattctttgcttcagaaaattaactagctaggatggtcagtgttgaagagatccgtaatgcacaacgtgcagagggccctgccactgtcatggctattggcaccgcaactcctccaaactgtgtcgatcagagtacctatcctgactattatttccgcatcaccaacagcgagcacatgaccgagctcaaagaaaaattcaagcgcatgtgtaagatatatatctctctcctttcttcatttctttatacaatatgtatattgcttattttcaacatattcctttgatttgattagtgatattaatgaaatttaatttattatttcgatcaggtgataagtcaatgattaagaagcgatacatgtacttaaatgaagaaatcctgaaagagaatccgagtgtttgtgcttacatggcaccttcgttggatgcaaggcaagacatggtggttgtggaggtaccaaagttgggaaaagaggctgcaactaaggcaatcaaggaatggggtcaacccaagtccaagattacccatctcatcttttgcaccactagtggtgtcgacatgcctggtgctgattatcagctcactaaactattaggccttcgcccctccgtcaagcgttacatgatgtaccaacaaggctgctttgccggtggcacggtgcttcgtttggccaaagacctcgctgaaaacaacaagggtgctcgcgtgcttgtcgtttgttctgagatcaccgcagtcacattccgcggcccaactgacacccatcttgatagccttgtgggtcaagccttgtttggagatggtgcagccgctgtcattgttggatcagaccccttaccagttgaaaagcctttgtttcagcttgtctggactgcccagacaatccttccagacagtgaaggggctattgatggacaccttcgcgaagttggtctcactttccatctcctcaaggatgttcctggactcatctccaagaatattgagaaggccttggttgaagccttccaacccttgggaatctccgattacaattctatcttctggattgcacaccctggtggacccgcaattttggaccaagttgaggctaagttaggcttgaagcctgaaaaaatggaagctactagacatgtgctcagcgagtatggtaacatgtcaagtgcatgtgtgctattcatcttggatcaaatgaggaagaaatcaatagaaaatggacttggcacaaccggtgaaggccttgactggggtgtgctatttggtttcggccctggactcaccgttgagactgttgtgctccgcagtgtcactgtctaa

SEQ ID NO: 30 - DNA, 3056 bp, Glycine max
aatccaatgaacaattttttttttcttgagatgcaaaggatagaaatattattaaaagcagcttagctgcacaagttgtgcccctactgaaagttatatcagaatacaattttgctgtgcatcaaagctatctaccagcaacatgttgcataattacgaaaaaaatagctttatcttgctgaacttggcagagcctctaaacatttacgaataattcagccatccgattatagaacataaccacgagaaaataaaagcaaccacctatcgctatcagcaaaccaaatgaagcaccagttgaacgccttgtattcccaccaccgaataccaaaattgaataaatttgattttgagaagaaaaaaattgcttattattattgttgatgtgaaatccacagaacctgtcacacataaacccattccaaacactgatgcttcccaaattgcattcgatgaaaatgctgacccaatcatccttaaagtttcccaaaggttttctcgatctcactaaattaaattcaacaggaattaggttgggttttaatttttatatctgtctttattaatcggataagtgaacaaattcgtaaaagtaaaagcgtaagacgctgttaatttagtcttgaaagtgctaaaatgaccaaaggtaactcattttaatccaaagtttcaaaggtcatgcgatgtaaatgacaataataacatactttcgaaattaaaatataatatagttaaattaaatttaaatactaaaataacaacataggaaattttaggaacattttttgttttcttgagaaaataaactttgttgttatttcattttaaatacaaacctaattatttaacaagagaatctatgtttgattttccaccgtatgtagttacacataggcttaattgcactttttatttttaattttttaatttttagtgaattttatccctaattttttaatttgacacattttattttcaatttttaagaaacttgtgaattttatcctctattatttatctatttataagcacaaaagttggggtaaaatttgtcaaaaaaaattaaaattaaggataaattttgtcaaaaaaatttaaagttggaataaaatttggcaaaaactaataagttagggataaaaaaaatataattatgtaactagtaaagtgatgaaggataaaatttgtaggattattaaaagttgagataaaatgtccaaaatttaaagattaagataaaattcgtcaaaaattaaaaaattagaataaaaaatataattaaatctaatgtttagtttatctataagaaaaatttcaaacctgaccccatcttattgcaatgcataatggagtgggtcagtccttccataggatcaccctggaggccacccccctttttttttccctctatgaccttcaccattgacttttcctaatcatcaattcatcactttcgtggcttctcctaatgaaaacgtgttgattaaaaaataaacaaaaaaccaaaaatattgggttgttaaaataagagagtagtcatcagtctacgtagccatgcggggcaccacatagttgaaacaaagcgcagccacgagtcagaggaagcatgcatagcatctacgtaccttagcctacctaccaatatcaactatctatatatatccacctttccaaatcactttccaacatccacccccatcatcatatcataccctttctatcctacttgctacttcccacttccattcttttcttaaccagctaggatggtgagtgttgaagagattcgtaaggcgcaacgtgcagaaggccctgccactgtcatggctattggcaccgccactcctcccaactgcgtggatcagagtacctatcctgactattatttccgcatcaccaacagcgagcacatgaccgagctcaaagaaaaattcaagcgcatgtgtaagatatatatctctctcctttcttcatttctttatacaatatgtatattgtttattttcaacatattcctttgatttgattagtgatattaatgaaatttaatttattatttcgatcagggtgataagtcgatgattaagaagcgatacatgtacttaaacgaagagatcctgaaagagaatccgagtgtttgtgcttacatggcaccttcgttggatgcaaggcaagacatggtggttgtggaggtaccaaagttgggaaaagaggctgcaactaaggcaatcaaggaatggggtcaacccaagtccaagattacccatctcatcttttgcaccactagtggtgtcgacatgcctggtgctgattatcagctcactaaactattaggccttcgcccctccgtcaagcgttacatgatgtaccaacaaggctgctttgccggtggcacggtgcttcgtttggccaaagacctcgctgaaaacaacaagggtgctcgcgtgcttgtcgtttgttctgagatcaccgcagtcacatttcgcggcccaactgacacccatcttgatagccttgtgggtcaagccttgtttggagatggtgcagccgctgtcattgttggatcagaccccttaccagttgaaaagcctttgtttcagcttgtctggactgcccagacaatccttccagacagtgaaggggctattgatggacaccttcgcgaagttggtctcactttccatctcctcaaggatgttcctggactcatctccaagaatattgagaaggccttggttgaagccttccaacccttgggaatctccgattacaattctatcttctggattgcacaccctggtggacccgcaattttggaccaagttgaggctaagttaggcctgaagcctgaaaaaatggaagctactagacatgtgctcagcgagtatggtaacatgtcaagtgcatgcgtgctattcatcttggatcaaatgaggaagaaatcaatagaaaatggacttggcacaaccggtgaaggtcttgactggggtgtgctatttggtttcggccctggactcaccgttgagactgttgtgctccgcagtgtcactctctga

SEQ ID NO: 31 - DNA, 3141 bp, Glycine max
aaaaaaaaattatatatttattattaatttaatttaaagtatattatacgttcaagagctaaatacatattcatcgacttattttaaaattgaagacttaattactttttgtcttgctacttatttatttaatttaattttttggtacaattactaataaagattcaatttgatttcttaattttaaaagcaatgaattttgattccttaattttcacaaaaggtgtcgttattatttaaaattaacgatggattaaaactgtcagctaatcataatcctcaaaaccgtgttcaatgacctgaagttaatctgaaagaaaggaaccaaattccatcattttataaaaattaaggaagcaaattgtattttttattaacagtggaacgaaattacacaaattaaataaatagtaatagtaaaaaaataattaaaccaaatttaaatcaattaaactctctccccctttctccaacaaacttgagcggctagtcttttttgtctcctttttcttcctttgttttgttcccacttgaaaattgcagcccacaaaaaaaataaaactaacccttcaaattaaacacaatacacaaaaatcccccgtagcattttttttcatatacataaaagctaacatgtaactcaaaagtacaagttttaaaagtcatcatatttaaagtcatcttattcaaccattatatatacatgtgaatcaactgaaacgtgattcttttaacttttaggatagagaataattttggtctagacatagaaaagagagacatcttcttcagatcaacacatgctaattagtaaacaattatttttaaaaacactaaaaaaaaaaggtatctttctctccaattttccattaggagaaccaaagactcaaagtgctctcttacaattactagaaaattctagtaaccggagaagatcctaaaattatgagtaacaattgttgagggaaagggggagaaacaataattttttagactagatcacaaatatttttttacaataagaaattctattcaaaatgaataagattattatgattagtaaaactcttactctaagtatttaacatagttacaggattcgttcgaaacttctccttaaactacaacaatctcacatcatttaatccacttgtttggtgctaagaaagtgtaatttgtggactcgttagaaaaataaataaataaataaatagtaaataaaagggtaggtataactacaactataagggaaaagtcaaaacagtctacttagttatgcggtacaccacatgtttgaaagaaaagcgcagtcagaggaagcatgcacgcgtctaccttaacggggaacctacccacccttttcagttatgtatatatatccaacattccaagacactttccacatccatttcccatcatcatacacttttctttcgtagctagctactccttaattactaattagtttcattctttggtgcaagctagcttcattagttgattcataaaattataacaatggtgagtgttgaagcaatccgtaaggcacaacgtgcagaaggccctgccaccgtcatggccatcggcactgccactcctccaaactgcgtcgatcagagtacttatcctgactattatttccgcatcaccaacagtgagcacatgactgagctcaaagaaaagttcaagcgcatgtgtaagatttatatctctctcttttatcctatcttcatttcagtatactatataatatgtatattgtttattttcaacatacaccatttatttgattaataatacatactaatgatatttaacttttttatttcgatcagatggtgagtgttgaagcaatccgtaaggcacaacgtgcagaaggccctgccaccgtcatggccatcggcactgccactcctccaaactgcgtcgatcagagtacttatcctgactattatttccgcatcaccaacagtgagcacatgactgagctcaaagaaaagttcaagcgcatgtgtaagatttatatctctctcttttatcctatcttcatttcagtatactatataatatgtatattgtttattttcaacatacaccatttatttgattaataatacatactaatgatatttaacttttttatttcgatcaggtgataagtcgatgattaagaagctatacatgtacttaaacgaagagatcctgaaggagaatcccagtgtttgtgcatatatggcaccttcgttggatgcaaggcaagacatggtggttgtggaggtaccaaagttgggaaaagaggctgcaactaaggcaatcaaggaatggggtcaacccaagtccaagattacccatctcatcttttgcaccactagtggtgtcgacatgcctggtgctgattatcagctcactaaactattaggccttcgtccctccgtcaagcgttacatgatgtaccaacaaggctgctttgccggtggcacggtgcttcgtttggccaaagacctcgctgaaaacaacaagggtgctcgcgtgcttgtcgtttgttctgagatcaccgcagtcacattccgcggcccaactgacacccatcttgatagccttgtgggtcaagccttgtttggagatggtgcagccgctgtcattgttggatcagaccccttaccagttgaaaagcctttgtttcagcttatctggactgcccaaacaatccttccagacagtgaaggggctattgatggccaccttcgcgaagttggactcactttccatctcctcaaggatgttcctggactcatctctaagaatattgagaaggccttggttgaagccttccaacccttgggaatctccgattacaattctatcttctggattgcacaccctggtggacccgcaattttggaccaagttgaggctaagttaggcttgaagcctgaaaaaatggaagctactagacatgtgctcagcgagtatggtaacatgtcaagtgcatgtgtgctattcatcttggatcaaatgaggaagaaatcaatagaaaatggacttggcacaaccggtgaaggccttgactggggtgtgctatttggtttcggccctggactcaccgttgagactgttgtgctccgcagtgtcactgtctaa

SEQ ID NO: 32 - DNA, 3104 bp, Glycine max
aggataataaaaaatcggttaagtggtttggacacttccaaagaagccacaagaagcacggttaagggagagttaaaatgaagtcgtcaaggagatctatgataaacaatatttctaaaactttaatttttaatccatccgaatgagtcgtcgtactgtctgtgtgattaaggtaacccctaaaccttaagtacaacgatcaatgtatggctcctcactcagtttgagtacacggatcaaaagttctctctatgatttttttgccagattttgtgctcaatcatctggcataatattttgataatccctccctccatgaacggatcttgttttttcactaattatctcccggttatctttgaaatgttcaccgtaacaccaccatgtttatctaatataggaagcaataagcctatatatttagctttacggtaaaaataaattcagctacaatgtataaaggatgaagaaaggaaagggataaaagacatggatttattatttttagacttttgatctctatcactctgatgagagagtgtaatgttttatcttacgcatgcgcaacttttcttttatctctgtcacttttacaggagtggttgctaatatgtgttttacaagagtgaatttcgtaatggattgtaaatcagtgaatgaagcatggtcttactcacacaaagcatgaaacatggtcttacttacataccaaagaataaaaagctattttcatgacattatgtggtcttactcacacaaagcatacctagcttgtcttacacacacaaagcatacctacaattattgagctaaattaacatttcatgacattattgtagtccactgtaacaaactcgccgcaatagcgagaaatttgtagtgctagttaagtgtcacttttcatgacatggattggatatagagttttcttgtcaattactttctttttttttgactttgatgtacaggtcttgaccaacctttagtaataatagtatcattcgtaattaaaaaaagaagaagtaaacttctattttttataataaaaaggactaaatatattttaggttgttataagttagaattaatttttaaactttgcacttagtttctaataaaaaaattcttgacttttggttctgaaattatattacattttgtacaaagaaaattctaagtcaagggggactaagttaattgtcacaagtgacaactctccttacacaattaagccataaacctggtttcagacagttctatagtccaatttataatcaaacacaaatgaaattggataaaagctattcactttgcaattgtatagatcaataatgtgtaagcttaattgcatttataacatgacatatttttatttactagaatacataaagaaccatgtgaggaaggcagggaaaaaggcaaaatagagtacactttaatttcaacctgaataggtaagaataaataagaaaaataaaaaggatttgtggttttgcacaatatatatatatatatatatatatatatatatatatatatatatatatatatggattcaacaaggctatcaatcaacagtcaacatagtcatgcagtgtacaatatagttgagagaaaacacagaacacagccaattcgttagaggaaacatgctcatcatctactcagtactcacctacccacttcaagttcaactgtctatctattcatatatatatacccacccttccaaaccactttgcaacatccatccaagccttttctttcctagctactacactttcattctttgcttcagaaaattaactagctaggatggtcagtgttgaagagatccgtaatgcacaacgtgcagagggccctgccactgtcatggctattggcaccgcaactcctccaaactgtgtcgatcagagtacctatcctgactattatttccgcatcaccaacagcgagcacatgaccgagctcaaagaaaaattcaagcgcatgtgtaagatatatatctctctcctttcttcatttctttatacaatatgtatattgcttattttcaacatattcctttgatttgattagtgatattaatgaaatttaatttattatttcgatcaggtgataagtcgatgattaagaagcgatacatgtacttaaatgaagaaatcctgaaagagaatccgagtgtttgtgcttacatggcaccttcgttggatgcaaggcaagacatggtggttgtggaggtaccaaagttgggaaaagaggctgcaactaaggcaatcaaggaatggggtcaacccaagtccaagattacccatctcatcttttgcaccactagtggtgtcgacatgcctggtgctgattatcagctcactaaactattaggccttcgcccctccgtcaagcgttacatgatgtaccaacaaggctgctttgccggtggcacggtgcttcgtttggccaaagacctcgctgaaaacaacaagggtgctcgcgtgcttgtcgtttgttctgagatcaccgcagtcacattccgcggcccaactgacacccatcttgatagccttgtgggtcaagccttgtttggagatggtgcagccgctgtcattgttggatcagaccccttaccagttgaaaagcctttgtttcagcttgtctggactgcccagacaatccttccagacagtgaaggggctattgatggacaccttcgcgaagttggtctcactttccatctcctcaaggatgttcctggactcatctccaagaatattgagaaggccttggttgaagccttccaacccttgggaatctccgattacaattctatcttctggattgcacaccctggtggacccgcaattttggaccaagttgaggctaagttaggcttgaagcctgaaaaaatggaagctactagacatgtgctcagcgagtatggtaacatgtcaagtgcatgtgtgctattcatcttggatcaaatgaggaagaaatcaatagaaaatggacttggcacaaccggtgaaggccttgactggggtgtgctatttggtttcggccctggactcaccgttgagactgttgtgctccgcagtgtcactgtctaa

SEQ ID NO: 33 - DNA, 3141 bp, Glycine max
tttatctttatgttttttttctctctattttaaattaaatttaaatatttctttaaaatatcagtagttaaaaataaaccttatatcacaatttaaattatttattatgaatctgaaatataatttatatattcaaaatatttgtttgttaagattttaattataatgtaatttaatattatgataaaataataaaactataccaactttgcaattcccgatcagattgttgttcgttggagcatactaaagcgccgcccaaaatatttgtttattaaaattttaattataatgtaatttaatattaagattatttatgtcagaaattttagttattatataaaataaatatttatactatgtaatactagttattaatgaaaatgaaagtaaaactatcgtgtagcataagtcaaataacaaagatcaatagataaagtcattttaagattaaaacttaaaagttccatttgttgtcaaagtcaatattgaccctgttttagttcttctttctcgcatgatatacttgaatgcaatgcaccttctcgtaaaagaaaagaataacaaaaacagtgaacttacaaaagctaaaagtaattagttataataaagccaactatataattttccacaaatcaaatatatttatttcatgaaattaatcataaaacaaacattttggtgatggttttatttatgcgtcttacaaattgaagaaagaaagcgatataattatgaattaaattaaaaatatactatatatttaatgttcaattttgattttggagaagttagatgactgaacttgttaagaagttgtgggatataagttacttttaacttagagccaaaaatgattcatttgatgttcatatttcattctgaaagtagacttgcatcaagttaacttaagataaaataataaaactataccaactccccaattcctgatcagattgttgttcgttggagcatactaacgtaaagcttcatcacccacttattccaaagataaagttcagtttaatcccctcccaaaccaaataaattatgaagtagttcacagccacacatgtctataatctcaaactaatatttatataacacatattaaaaattattaatttatgattacttgattatatattacataaaaattaatatagtgtaagaaccaagataaatcataatcatttaataatttctcttcagaccaacataaccacgaccagtttctttcatgagagagaagataagagaaaaaatgtttttcaattttttttaaaaaagaatttaatattagtctttgaaatttttaagcaccatggaggtgaaaaaaatagatatccatataatggacaggatatctgaattgcaaaaaaatcatgaatctcttgtttaaaaacagttttatttaaaacatttattttttattggaatgttttcaagatgataaatgagacaaatcaatcaatcagacttggtattaaaaacaaataatttcctcgtgacattttttttttcataaacataactcaactaaagaaaaaaaaacagaaaattaaaacccggttatttgctgatcattaggaaaagaaaaaaaaatgggttggtaagtataactataatggggagaatcagcggtctacttagacatgcggtgggtgcacaccacaagcgcagtcagagaaaggaagcatgcactgcatctaccttaatctacctacccacacttttctatatatatatatccacccttccaagccactttgcaacatccatccaagccttttctttcgtagatagctactacttcactttcatcctttgctccagaaaattaactagctaggatggtgagtgttgaagagattcgtaaggcgcaacgtgcagaaggccctgccactgtcatggctattggcaccgccactcctcccaactgcgtggatcagagtacctatcctgactattatttccgcatcaccaacagcgagcacatgaccgagctcaaagaaaaattcaaacgcatgtgtaagatatctctctcttttatcctatcttcatttcattatataatatgcatgttgcttatttccaacatatacctttgatttcattaatgatatcaatgaaatttaatttattatttcaggtgataagtcgatgattaagaagcgatacatgtacttaaacgaagagatcctgaaggagaatcccagtgtttgtgcatatatggcaccttcgttggatgcaaggcaagacatggtggttatggaggtaccaaagttgggaaaagaggctgcaactaaggcaatcaaggaatggggtcaacccaagtccaagattacccatctcatcttttgcaccactagtggtgtcgacatgcctggtgctgattatcagctcactaaactattaggccttcgtccctccgtcaagcgttacatgatgtaccaacaaggctgctttgccggtggcacggtgcttcgtttggccaaagacctcgctgaaaacaacaagggtgctcgcgtgcttgtcgtttgttctgagatcaccgcagtcacattccgcggcccaactgacacccatcttgatagccttgtgggtcaagccttgtttggagatggtgcagccgctgtcattgttggatcagaccccttaccagttgaaaagcctttgtttcagcttgtctggactgcccagacaatccttccagacagtgaaggggctattgatggacaccttcgcgaagttggtctcactttccatctcctcaaggatgttcctggactcatctccaagaatattgagaaggccttggttgaagccttccaacccttgggaatctccgattacaattctatcttctggattgcacaccctggtggacccgcaattttggaccaagttgaggctaagttaggcttgaagcctgaaaaaatggaagctactagacatgtgctcagcgagtatggtaacatgtcaagtgcatgtgtgctattcatcttggatcaaatgaggaagaaatcaatagaaaatggacttggcacaaccggtgaaggccttgactggggtgtgctatttggtttcggccctggactcaccgttgagactgttgtgctccgcagtgtcactgtctaa

SEQ ID NO: 34 - DNA, 4808 bp, Glycine max
  misc_feature (1)..(4808): n= a, t, c, or g
cctatactctggcatgttctcctgtgtaatctttaattgctggatcttcttcatatttgattacaagattatagtaggagctatgaatgaagttgattcagaattatactagaatttttataattttttgtttcgtttcatgttttgataaatgtttatttatttaatattaactggtatacacacatctcatgccctaactcctatatacacacctgttgttacccataccaatgtgatgataatgggagtgagcatttgcaaacaatgcccattcacaactttcaattctgtttactagagttctttagtaagttgtttaaccacgagacataacatttgtcttattttatagttactaagttcaactatttatattgtctttcacttgcaaccatgtttatccctatattaatttgtaattatcaaatgttgcccgatgataaatttggccccaaatattccaatttcctgtactttttctccggtagaagtttccattatttttaaaatcttacacaaacatgattcagtttggataaaatttcttaacaagcatttataggtaaagaaaataaggaagcagaataaatcgattttcaattttgattttggagaagttagatgactgaacttgttaagaagttgtgggatataagttacttttaacttagagccaaaaatgattcatttgatgttcatatttcattctgaaagtagacttgcatcaagttaacttaagataaaataataaaactataccaactccccaattcctgatcagattgttgttcgttggagcatactaacgtaaagcttcatcacccacttattccaaagataaagttcagtttaatcccctcccaaaccaaataaattatgaagtagttcacagccacacatgtctataatctcaaactaatatttatataacacatattaaaaattattaatttatgattacttgattatatattacataaaaattaatatagtgtaagaaccaagataaatcataatcatttaataatttctcttcagaccaacataaccacgaccagtttctttcatgagagagaagataagagaaaaaatgtttttcaattttttttaaaaaagaatttaatattagtctttgaaatttttaagcaccatggaggtgaaaaaaatagatatccatataatggacaggatatctgaattgcaaaaaaatcatgaatctcttgtttaaaaacagttttatttaaaacatttattttttattggaatgttttcaagatgataaatgagacaaatcaatcaatcagacttggtattaaaaacaaataatttcctcgtgacattttttttttcataaacataactcaactaaagaaaaaaaaacagaaaattaaaacccggttatttgctgatcattaggaaaagaaaaaaaaatgggttggtaagtataactataatggggagaatcagcggtctacttagacatgcggtgggtgcacaccacaagcgcagtcagagaaaggaagcatgcactgcatctaccttaatctacctacccacacttttctatatatatatatccacccttccaagccactttgcaacatccatccaagccttttctttcgtagatagctactacttcactttcatcctttgctccagaaaattaactagctaggatggtgagtgttgaagagattcgtaaggcgcaacgtgcagaaggccctgccactgtcatggctattggcaccgccactcctcccaactgcgtggatcagagtacctatcctgactattatttccgcatcaccaacagcgagcacatgaccgagctcaaagaaaaattcaaacgcatgtgtaagatatctctctcttttatcctatcttcatttcattatataatatgcatgttgcttatttccaacatatacctttgatttcattaatgatatcaatgaaatttaatttattatttcaggtgataagtcgatgattaagaagcgatacatgtacttaaacgaagagatcctgaaggagaatcccagtgtttgtgcatatatggcaccttcgttggatgcaaggcaagacatggtggttatggaggtaccaaagttgggaaaagaggctgcaactaaggcaatcaaggaatggggtcaacccaagtccaagattacccatctcatcttttgcaccactagtggtgtcgacatgcctggtgctgattatcagctcactaaactattaggcctagtacctccgtcaagcgttacatgatgtaccaacaaggctgctttgccggtggcacggtgcttcgtttggccaaagacctcgctgaaaacaacaagggtgctcgcgtgcttgtcgtttgttctgagatcaccgcagtcacattccgcggcccaactgacacccatcttgatagccttgtgggtcaagccttgtttggagatggtgcagccgctgtcattgttggatctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccaccgtatgtagttacacataggcttaatttcactttttattgttaatctttttaatttttagtgaattttatccctaattttttaatttgacacattttattttcaatttttaagaaacttgtgaattttatcctctattatttatctatttataagcacaaaagttgggggaaaatttggcaacctcantaaaagtgaggataaattctgtcaaaaaaatttaaagttggaataaaatttggcaaaaactaataagttagggataaaaaaaatataattatgtaactagcaaagtgatgaaggataaaatttgtaggattattaaaagttgagataaaatgtccaaaatttaaagattaagataaaattcgtcaaaaattaaaaaattagaataaaaaatataattaaatctaatgtttagtttatctataagaaaaatttcaaacctgaccccatcttattgcaatgcataatggagtgggtcagtccttccataggatcaccctggaggccacccccctttttttttccctctatgaccttcaccattgacttttcctaatcatcaattcatcactttcgtggcttctcctaatgaaaacgtgttgattaaaaaataaacaaaaaaccaaaaatattgggttgttaaaataagagagtagtcatcagtctacgtagccatgcggggcaccacatagttgaaacaaagcgcagccacgagtcagaggaagcatgcatagcatctacgtaccttagcctacctaccaatatcaactatctatatatatccacctttccaaatcactttccaacatccacccccatcatcatatcataccctttctatcctacttgctacttcccacttccattcttttcttaaccagctaggatggtgagtgttgaagagattcgtaaggcgcaacgtgcagaaggccctgccactgtcatggctattggcaccgccactcctcccaactgcgtggatcagagtacctatcctgactattatttccgcatcaccaacagcgagcacatgaccgagctcaaagaaaaattcaagcgcatgtgtaagatatatatctctctcctttcttcatttctttatacaatatgtatattggttattttcaacatattcctttgatttgattagtgatattaatgaaatttaatttattatttcgatcaggtgataagtcgatgattaagaagcgatacatgtacttaaacgaagagatcctgaaagagaatccgagtgtttgtgcttacatggcaccttcgttggatgcaaggcaagacatggtggttgtggaggtaccaaagttgggaaaagaggctgcaactaaggcaatcaaggaatggggtcaacccaagtccaagattacccatctcatcttttgcaccactagtggtgtcgacatgcctggtgctgattatcagctcactaaactattaggccttcgcccctccgtcaagcgttacatgatgtaccaacaaggctgctttgccggtggcacggtgcttcgtttggccaaagacctcgctgaaaacaacaagggtgctcgcgtgcttgtcgtttgttctgagatcaccgcagtcacatttcgcggcccaactgacacccatcttgatagccttgtgggtcaagccttgtttggagatggtgcagccgctgtcattgttggatcagaccccttaccagttgaaaagcctttgtttcagcttgtctggactgcccagacaatccttccagacagtgaaggggctattgatggacaccttcgcgaaggtggtctcactttccatctcctcaaggatgttcctggactcatctccaagaatattgagaaggccgtggttgaagccttccaacccttgggaatctccgattacaattctatcttctggattgcacaccctggtggacccgcaattttggaccaagttgaggctaagttaggcctgaagcctgaaaaaatggaagctactagacatgtgctcagcgagtatggtaacatgtcaagtgcatgcgtgctattcatcttggatcaaatgaggaagaaatcaatagaaaatggacttggcacaaccggtgaaggtcttgactggggtgtgctatttggtttcggccctggactcaccgttgagactgttgtgctccgcagtgtcactctctga

SEQ ID NO: 35 - DNA, 3114 bp, Glycine max
caattatattactgcctcacttctaagacaatgatattttaaacctgtgacccactaattcacaaacatttaattgatataaattttaaataaaatattctcaatttattaactcattttgttataagctaattatcccattagccatcaataacaataaattttactattcatcgactattttttttatgataaatgtctcttttaattgcatgtgttaattgatctttttaattatgcttaagaatagtatttaaaaaatagtttaaaaagctaaaaagattattgttttgaaaaaaaatagaaagaccatttgttttaggaaggagggagtattatatgcaatagtctgtttatcattaaatgaatattaatttttgttacaattttttataagtcgtgttttttttactattttttaaatgaaaaatgaataatttaatacattctcaactttttttatatttagtttagtgtagtgaaattaagcacaatttcaccttttttttaaattgtttaaaattcacgactccgcattatattataatatattgtgttaatattattagtaaataattttttctcatttactatttggttgagagaataaggttatattattagcaaatgcattatttgacaaattttaattaagttcctaaattattttttttcaattgttctcttaacttatatttttttaaatgatgttcctaaactattaggaataaatgtatatgtccaagaatcaatctgtcatgtaactaattaggaataaatattattagaatttgatcatcatgtactactataaaacaattgattggataatatctttaattaaaatcatggactcattatcataaactagtattgtataaatttaatccaaattaatcttgattataaaaaacaagagacatccaaattcaaaaaataatagcatttattaaataaagattaataaatttcatttattaaattacacatatagatgatatatatgtgaatataattctaaaagttaataacattactttaaattatcaataaaaaattcataagaaaaaaaaaataattttgttttacttaaaattatcataataattaataagttctttattatattttaattttggacatcttctatctattttttaaacaagatacccaatatcttaaggtattagttgaatagttattaagtaatgactaatgagtctgagttttatttaaaacaattattttttcgaattatttttctgggcgataaatgaacttaaactaatcatttacgcacaatattaaaacaagtaaatctctcgtgacatttctttttgatacacttgaaactgatcaaaactaatttcttaccagggatatgagtccctttcattcacatcaacacacataacagtaagtaattatttttccaaaaactctaaccagaaataaaaaagtaattccaaaattaggagaagcaattgtaaagaagtatggactatggagaacaaaaaaaaaatttgctgattattgggggaaaagaatgggttggtgtgttgggagagtcaacagtctacttagacatgcggtacatacaccatatatttgaaagaaaaaaaagcgtagtcagaggaagcatgcgcgcatctacctacccacccttttcaattatgcatgtatatatatatctgagccactttgccacattcattcccaccctcatacccttttctttcgtgcctagctactccttaattactttcattctttaatttgctgcaagctatagcttcattagttcattcacaaaattaattattacaatggtgagtgttgaagagatccgtcaggcacaacgtgcagaaggccctgccactgtcatggctattggcaccgccactcctcccaactgcgtggatcagagtacctatcctgactattatttccgcatcaccaacagcgagcacatgaccgagctcaaagaaaaattcaaacgcatgtgtaagatatctctctcttttatcctatcttcatttcattatataatatgcatgttgcttatttccaacatatacctttgatttcattaatgatatcaatgaaatttaatttattatttcaggtgataagtcgatgattaagaagcgatacatgtacttaaacgaagagatcctgaaggagaatcccagtgtttgtgcatatatggcaccttcgttggatgcaaggcaagacatggtggttatggaggtaccaaagttgggaaaagaggctgcaactaaggcaatcaaggaatggggtcaacccaagtccaagattacccatctcatcttttgcaccactagtggtgtcgacatgcctggtgctgattatcagctcactaaactattaggccttcgtccctccgtcaagcgttacatgatgtaccaacaaggctgctttgccggtggcacggtgcttcgtttggccaaagacctcgctgaaaacaacaagggtgctcgcgtgcttgtcgtttgttctgagatcactggagtcacattccgcggcccaactgacacccatcttgatagccttgtgggtcaagccttgtttggagatggtgcagccgctgtcattgttggatcagaccccttaccagttgaaaagcctttgtttcagcttgtctggactgcccagacaatccttccagacagtgaaggggctattgatggacaccttcgcgaagttggtctcactttccatctcctcaaggatgttcctggactcatctccaagaatattgagaaggccttggttgaagccttccaacccttgggaatctccgattacaattctatcttctggattgcacaccctggtggacccgcaattttggaccaagtggaggctaagttaggcttgaagcctgaaaaaatggaagctactaggcatgtgctcagcgagtatggtaacatgtcaagtgcatgtgtgctattcatcttggatcaaatgcggaagaaatcaatagaaaatggacttggcacaaccggcgaaggccttgactggggtgtgctatttggtttcggtcctggactcactgttgagactgttgtactccgcagtgtcactgtctaa

SEQ ID NO: 36 - DNA, 2961 bp, Glycine max
atcactttactagttacataattatattttttttatccctaacttattagtttttgccaaattttattccaactttaaatttttttgacaaaatttatccttaattttaattttttttgacaaattttaccccaacttttgtgcttataaatagataaataatagaggataaaattcacaagtttcttaaaaattgaaaataaaatgtgtcaaattaaaaaattagggataaaattcactaaaaattaaaaaattaaaaataaaaagtgcaattaagcctatgtgtaactacatacggtggaaaatcaaacatagattctcttgttaaataattaggtttgtatttaaaatgaaataacaacaaagtttattttctcaagaaaacaaaaaatgttcctaaaatttcctatgttgttattttagtatttaaatttaatttaactatattatattttaatttcgaaagtatgttattattgtcatttacatcgcatgacctttgaaactttggattaaaatgagttacctttggtcattttagcactttcaagactaaattaacagcgtcttacgcttttacttttacgaatttgttcacttatccgattaataaagacagatataaaaattaaaacccaacctaattcctgttgaatttaatttagtgagatcgagaaaacctttgggaaactttaaggatgattgggtcagcattttcatcgaatgcaatttgggaagcatcagtgtttggaatgggtttatgtgtgacaggttctgtggatttcacatcaacaataataataagcaatttttttcttctcaaaatcaaatttattcaattttggtattcggtggtgggaatacaaggcgttcaactggtgcttcatttggtttgctgatagcgataggtggttgcttttattttctcgtggttatgttctataatcggatggctgaattattcgtaaatgtttagaggctctgccaagttcagcaagataaagctatttttttcgtaattatgcaacatgttgctggtagatagctttgatgcacagcaaaattgtattctgatataactttcagtaggggcacaacttgtgcagctaagctgcttttaataatatttctatcctttgcatctcaagaaaaaaaaaattgttcattggattggagtcgattttagttttgccagaaataactgaatcaatccaaatcaaattgaattactaaatactattaacattaaagctactttgttgatgatgttgatacgatacactccctttttataatgtcaatgactatatcctttctctgtcaacaaatgactatgtccttttatccaaatctatttatttgagaatcattttaacgtgtttttaatcaaatttgtaaggtatatatataatcattataatgggatagtcaacagtcaacatagtcatgcagtgtacaatatagttgagagaaaacacagaacacagccaattcgttagaggaaacatgctcatcatctactcagtactcacctacccacttcaagttcaactgtctatctattcatatatatatacccacccttccaaaccactttgcaacatccatccaagccttttctttcctagctactacactttcattctttgcttcagaaaattaactagctaggatggtcagtgttgaagagatccgtaatgcacaacgtgcagagggccctgccactgtcatggctattggcaccgcaactcctccaaactgtgtcgatcagagtacctatcctgactattatttccgcatcaccaacagcgagcacatgaccgagctcaaagaaaaattcaagcgcatgtgtaagatatatatctctctcctttcttcatttctttatacaatatgtatattgcttattttcaacatattcctttgatttgattagtgatattaatgaaatttaatttattatttcgatcaggtgataagtcaatgattaagaagcgatacatgtacttaaatgaagaaatcctgaaagagaatccgagtgtttgtgcttacatggcaccttcgttggatgcaaggcaagacatggtggttgtggaggtaccaaagttgggaaaagaggctgcaactaaggcaatcaaggaatggggtcaacccaagtccaagattacccatctcatcttttgcaccactagtggtgtcgacatgcctggtgctgattatcagctcactaaactattaggccttcgcccctccgtcaagcgttacatgatgtaccaacaaggctgctttgccggtggcacggtgcttcgtttggccaaagacctcgctgaaaacaacaagggtgctcgcgtgcttgtcgtttgttctgagatcaccgcagtcacattccgcggcccaactgacacccatcttgatagccttgtgggtcaagccttgtttggagatggtgcagccgctgtcattgttggatcagaccccttaccagttgaaaagcctttgtttcagcttgtctggactgcccagacaatccttccagacagtgaaggggctattgatggacaccttcgcgaagttggtctcactttccatctcctcaaggatgttcctggactcatctccaagaatattgagaaggccttggttgaagccttccaacccttgggaatctccgattacaattctatcttctggattgcacaccctggtggacccgcaattttggaccaagttgaggctaagttaggcttgaagcctgaaaaaatggaagctactagacatgtgctcagcgagtatggtaacatgtcaagtgcatgtgtgctattcatcttggatcaaatgaggaagaaatcaatagaaaatggacttggcacaaccggtgaaggccttgactggggtgtgctatttggtttcggccctggactcaccgttgagactgttgtgctccgcagtgtcactgtctaa

SEQ ID NO: 37 - DNA, 3142 bp, Glycine max
caaagtagctttaatgttaatagtatttagtaattcaatttgatttggattgattcagttatttctggcaaaactaaaatcgactccaatccaatgaacaattttttttttcttgagatgcaaaggatagaaatattattaaaagcagcttagctgcacaagttgtgcccctactgaaagttatatcagaatacaattttgctgtgcatcaaagctatctaccagcaacatgttgcataattacgaaaaaaatagctttatcttgctgaacttggcagagcctctaaacatttacgaataattcagccatccgattatagaacataaccacgagaaaataaaagcaaccacctatcgctatcagcaaaccaaatgaagcaccagttgaacgccttgtattcccaccaccgaataccaaaattgaataaatttgattttgagaagaaaaaaattgcttattattattgttgatgtgaaatccacagaacctgtcacacataaacccattccaaacactgatgcttcccaaattgcattcgatgaaaatgctgacccaatcatccttaaagtttcccaaaggttttctcgatctcactaaattaaattcaacaggaattaggttgggttttaatttttatatctgtctttattaatcggataagtgaacaaattcgtaaaagtaaaagcgtaagacgctgttaatttagtcttgaaagtgctaaaatgaccaaaggtaactcattttaatccaaagtttcaaaggtcatgcgatgtaaatgacaataataacatactttcgaaattaaaatataatatagttaaattaaatttaaatactaaaataacaacataggaaattttaggaacattttttgttttcttgagaaaataaactttgttgttatttcattttaaatacaaacctaattatttaacaagagaatctatgtttgattttccaccgtatgtagttacacataggcttaattgcactttttatttttaattttttaatttttagtgaattttatccctaattttttaatttgacacattttattttcaatttttaagaaacttgtgaattttatcctctattatttatctatttataagcacaaaagttggggtaaaatttgtcaaaaaaaattaaaattaaggataaattttgtcaaaaaaatttaaagttggaataaaatttggcaaaaactaataagttagggataaaaaaaatataattatgtaactagtaaagtgatgaaggataaaatttgtaggattattaaaagttgagataaaatgtccaaaatttaaagattaagataaaattcgtcaaaaattaaaaaattagaataaaaaatataattaaatctaatgtttagtttatctataagaaaaatttcaaacctgaccccatcttattgcaatgcataatggagtgggtcagtccttccataggatcaccctggaggccacccccctttttttttccctctatgaccttcaccattgacttttcctaatcatcaattcatcactttcgtggcttctcctaatgaaaacgtgttgattaaaaaataaacaaaaaaccaaaaatattgggttgttaaaataagagagtagtcatcagtctacgtagccatgcggggcaccacatagttgaaacaaagcgcagccacgagtcagaggaagcatgcatagcatctacgtaccttagcctacctaccaatatcaactatctatatatatccacctttccaaatcactttccaacatccacccccatcatcatatcataccctttctatcctacttgctacttcccacttccattcttttcttaaccagctaggatggtgagtgttgaagagattcgtaaggcgcaacgtgcagaaggccctgccactgtcatggctattggcaccgccactcctcccaactgcgtggatcagagtacctatcctgactattatttccgcatcaccaacagcgagcacatgaccgagctcaaagaaaaattcaagcgcatgtgtaagatatatatctctctcctttcttcatttctttatacaatatgtatattgtttattttcaacatattcctttgatttgattagtgatattaatgaaatttaatttattatttcgatcaggtgataagtcgatgattaagaagcgatacatgtacttaaacgaagagatcctgaaagagaatccgagtgtttgtgcttacatggcaccttcgttggatgcaaggcaagacatggtggttgtggaggtaccaaagttgggaaaagaggctgcaactaaggcaatcaaggaatggggtcaacccaagtccaagattacccatctcatcttttgcaccactagtggtgtcgacatgcctggtgctgattatcagctcactaaactattaggccttcgcccctccgtcaagcgttacatgatgtaccaacaaggctgctttgccggtggcacggtgcttcgtttggccaaagacctcgctgaaaacaacaagggtgctcgcgtgcttgtcgtttgttctgagatcaccgcagtcacatttcgcggcccaactgacacccatcttgatagccttgtgggtcaagccttgtttggagatggtgcagccgctgtcattgttggatcagaccccttaccagttgaaaagcctttgtttcagcttgtctggactgcccagacaatccttccagacagtgaaggggctattgatggacaccttcgcgaagttggtctcactttccatctcctcaaggatgttcctggactcatctccaagaatattgagaaggccttggttgaagccttccaacccttgggaatctccgattacaattctatcttctggattgcacaccctggtggacccgcaattttggaccaagttgaggctaagttaggcctgaagcctgaaaaaatggaagctactagacatgtgctcagcgagtatggtaacatgtcaagtgcatgcgtgctattcatcttggatcaaatgaggaagaaatcaatagaaaatggacttggcacaaccggtgaaggtcttgactggggtgtgctatttggtttcggccctggactcaccgttgagactgttgtgctccgcagtgtcactctctga